(19) United States
(12) Patent Application Publication
(10) Pub. No.: US 2024/0200016 A1
(43) Pub. Date: Jun. 20, 2024

(54) BIOPROCESS CONTROL SYSTEMS, MODULES, LIBRARIES AND GRAPHICAL USER INTERFACES

(71) Applicant: Life Technologies Corporation, Carlsbad, CA (US)

(72) Inventors: Robert Howe, Cork (IE); Sean Zimmerman, Encinitas, CA (US)

(21) Appl. No.: 18/493,350

(22) Filed: Oct. 24, 2023

Related U.S. Application Data
(60) Provisional application No. 63/418,924, filed on Oct. 24, 2022.
(60) Provisional application No. 63/428,591, filed on Nov. 29, 2022.
(60) Provisional application No. 63/521,320, filed on Jun. 15, 2023.

(51) Int. Cl. C12M 1/36; G06F 3/04847

(52) U.S. Cl. CPC C12M 41/48; G06F 3/04847; G06F 2203/04803

(57) ABSTRACT

Bioprocess control systems, computing devices, hardware architectures, modules, libraries and graphical user interfaces. More specifically, bioprocessing instrument control system modules, libraries and user interfaces used to control bioprocessing instruments and biological processes are disclosed. A computing device(s) having a display and functional graphical user interface configured to receive input from a user, includes a primary selection area with at least one virtual input to control operation of a bioprocessing instrument set to generate a cell based therapeutic and a secondary selection area with at least one virtual output indicating a process state and/or a device state of an instrument contained in a bioprocessing instrument set.

### CROSS-REFERENCE TO RELATED APPLICATIONS

This application claims the benefit of priority to U.S. Provisional Patent Application Ser. No. 63/418,924, filed Oct. 24, 2022; U.S. Provisional Patent Application Ser. No. 63/428,591, filed Nov. 29, 2022; and U.S. Provisional Patent Application Ser. No. 63/521,320, filed Jun. 15, 2023, the disclosures of which are considered part of, and are herein incorporated by reference in the disclosure of this application.

### BACKGROUND

### Field

The present disclosure relates to bioprocess control systems, modules, executable software libraries, and graphical user interfaces. More specifically, the present disclosure relates to bioprocessing instrument control system modules, executable software libraries and graphical user interfaces used to control bioprocessing instruments and biological processes, e.g., to generate a genetically modified cell.

### Background Information

Manufacturing of cellular therapy products typically involves multiple cell processing steps that require cumbersome manual operations performed in environments that fail to sufficiently mitigate contamination risks. Skilled laboratory technicians, adequate sterile enclosures such as cleanroom facilities, and associated protocols and procedures for regulated GMP manufacturing are expensive. Typical manufacturing processes employ numerous automated platforms to perform various steps of a standard workflow used to generate a cellular therapeutic.

However, the majority of industry efforts to automate cell therapy manufacturing have been directed to automating individual processing steps of a cell therapy manufacturing workflow without providing automated operational control of the entire workflow. As such, there remains a need for an automated cell therapy workflow in which individual automated platforms are integrated and may be operationally controlled using a minimal number of technicians. Previous attempts at automation of bioprocessing are addressed in various embodiments disclosed here.

### SUMMARY

The present disclosure provides systems, and components thereof, and methods for bioprocessing, such as production of cell-based products and therapeutics. Aspects and embodiments of the present disclosure achieve one or more advantages over conventional bioprocessing systems, including, for example, improved sterility, automation, lower cost of goods, lower labor costs, higher repeatability, higher reliability, lower risk of operator error, lower risk of contamination, higher process flexibility, higher capacity, higher instrument throughput, higher degree of process scalability, and shorter process duration.

In one aspect, the disclosure provides a bioprocess control system (BPCS) to be used in connection with a bioprocessing instrument set. The system includes a computing device having a display and a processor disposed in connection with a memory, the display having a graphical user interface for each bioprocessing instrument of the bioprocessing instrument set configured in input/output communication with each bioprocessing instrument, each graphical user interface having a primary selection area configured to receive a virtual input from a user and a secondary selection area having a virtual output. In embodiments, the memory includes computer-readable instructions executable by the processor, which when executed cause the computing device to communicate with a bioprocessing instrument of the bioprocessing instrument set to control operation of the bioprocessing instrument dependent on the virtual input, and display a virtual output in the primary selection area or the secondary selection area, the virtual output being dependent on data received from the bioprocessing instrument.

In another aspect, the disclosure provides a bioprocess control system (BPCS) that includes a computing device having a processor; and a memory storing computer-readable instructions executable by the processor, causing the computing device to transmit, to a client computing device, graphical display data of a virtual output indicating a process state or a device state of a bioprocessing instrument.

In still another aspect, the disclosure provides a bioproduction system including a bioprocessing instrument set. In embodiments, the system includes a bioprocessing instrument set and a computing device for controlling operation of the system including independently controlling operation of bioprocessing instruments of the instrument set. In embodiments, the computing device includes a processor; and a memory storing computer-readable instructions executable by the processor, causing the computing device to display, on a display of the computing device, a graphical user interface comprising a primary selection area having at least one virtual input controlling operation of the bioprocessing instruments. In embodiments, the bioprocessing instrument set includes one or more of a blood processing system, a magnetic bead processing system, an electroporation system, a freezing system, an incubator system, a bioreactor, and a fill and finish system.

In various embodiments, the computing device of the bioprocessing control system or the bioproduction system is configured in input/output communication with at least one secondary computing device, wherein the at least one secondary computing device has a memory storing instructions for controlling operation of one or more bioprocessing instruments of the bioprocessing instrument set, and optionally the computing device is communicatively configured in communication with one or more bioprocessing instruments through at least one intermediary controller and/or network switch.

In yet another aspect, the disclosure provides a computing device. In some embodiments, the computing device includes a processor; and a memory storing computer-readable instructions executable by the processor, causing the computing device to display, on a display of the computing device, a graphical user interface comprising a primary selection area configured to receive a plurality of virtual inputs to independently control operation of bioprocessing instruments in input/output communication with the computing device, and one or more secondary selection areas having one or more virtual outputs indicating a process state or a device state for one or more of the bioprocessing instruments. In some embodiments, the computing device includes a processor; and a memory storing computer-readable instructions executable by the processor, causing the computing device to display, on a display of the computing device, a graphical user interface comprising a primary selection area comprising at least one virtual input controlling the operation of a bioprocessing instrument and a secondary selection area comprising at least one virtual output indicating a process state or a device state of the bioprocessing instrument.

In another aspect, the disclosure provides a method of generating genetically modified cells using the bioprocess control system, bioproduction system and/or computing device of the disclosure. The method includes: providing a cell sample comprising live cells; preparing the cell sample by centrifugation; generating an enriched population of live cells by magnetic separation; genetically modifying the enriched population of live cells via a transfection process comprising electroporation to generate a population of genetically modified cells; incubating the population of genetically modified cells; and washing the incubated cells, wherein the method is performed under functionally-closed and sterile conditions. In some embodiments, the method further includes activating the enriched population of live cells by contacting the enriched population of live cells with a modulatory agent prior to genetically modifying the cells, optionally formulating a cellular therapy product comprising the washed cells, and optionally freezing the cellular therapy product.

### BRIEF DESCRIPTION OF THE DRAWINGS

Various embodiments of the present disclosure are described herein with reference to the appended drawings. It is appreciated that these drawings depict only typical embodiments of the disclosure and are therefore not to be considered limiting of its scope.

FIG. 1A depicts a block diagram of a bioprocess control system in embodiments of the disclosure.

FIG. 1B depicts a hardware architecture for a bioprocess control system in embodiments of the disclosure.

FIG. 2 depicts a schematic workflow diagram for controlling/monitoring the bioprocess control system in embodiments of the disclosure.

FIG. 3 depicts a hardware architecture for a bioprocess control system in embodiments of the disclosure.

FIG. 4A depicts a hardware architecture for a blood processing system in the bioprocess control system in embodiments of the disclosure.

FIG. 4B depicts a hardware architecture for a magnetic bead processing system in the bioprocess control system in embodiments of the disclosure.

FIG. 4C depicts a hardware architecture for an electroporation system in the bioprocess control system in embodiments of the disclosure.

FIG. 4D depicts a hardware architecture for a freezing system in the bioprocess control system in embodiments of the disclosure.

FIG. 4E depicts hardware architecture for an incubator system in the bioprocess control system in embodiments of the disclosure.

FIG. 5 depicts a flow chart for batch control data for the bioprocess control system in embodiments of the disclosure.

FIG. 6 depicts a flow chart for batch control data for the bioprocess control system in embodiments of the disclosure.

FIG. 7 depicts a schematic phase diagram for software modules for the bioprocess control system in embodiments of the disclosure.

FIG. 8 illustrates an overview of a graphical user interface displayed on a client computing device in embodiments of the disclosure.

FIG. 9A illustrates an overview of a graphical user interface displayed on a client computing device for a blood processing device in embodiments of the disclosure.

FIG. 9B illustrates a main display of a graphical user interface displayed on a client computing device for a blood processing device in embodiments of the disclosure.

FIG. 9C illustrates interface parameters of a graphical user interface displayed on a client computing device for a blood processing device in embodiments of the disclosure.

FIG. 9D illustrates an equipment module faceplate of a graphical user interface displayed on a client computing device for a blood processing device in embodiments of the disclosure.

FIG. 10A illustrates an overview of a graphical user interface displayed on a client computing device for a magnetic bead processing system in embodiments of the disclosure.

FIG. 10B illustrates an equipment module faceplate of a graphical user interface displayed on a client computing device for a magnetic bead processing system in embodiments of the disclosure.

FIG. 11A illustrates an overview of a graphical user interface displayed on a client computing device for an electroporation system in embodiments of the disclosure.

FIG. 11B illustrates an equipment module faceplate of a graphical user interface displayed on a client computing device an electroporation system in embodiments of the disclosure.

FIG. 12A illustrates an overview of a graphical user interface displayed on a client computing device for an incubator system in embodiments of the disclosure.

FIG. 12B illustrates a main display of a graphical user interface displayed on a client computing device for an incubator system in embodiments of the disclosure.

FIG. 12C illustrates an equipment module of a graphical user interface displayed on a client computing device in embodiments of the disclosure.

FIG. 13A illustrates an overview of a graphical user interface displayed on a client computing device for a freezer system in embodiments of the disclosure.

FIG. 13B illustrates a main display of a graphical user interface displayed on a client computing device for a freezer system in embodiments of the disclosure.

FIG. 13C illustrates an equipment module of a graphical user interface displayed on a client computing device for a freezer system in embodiments of the disclosure.

FIG. 14 illustrates a batch-run user interface displayed on a client computing device in embodiments of the disclosure.

FIG. 15 depicts a block diagram of an example computing device that can perform some or all operations of a bioprocess control system, client computing device, computing device, and/or controller in embodiments of the disclosure.

### DETAILED DESCRIPTION

This disclosure is not limited only to the specific and exemplified apparatus, systems, methods, or process parameters disclosed herein. The exemplary apparatus, systems, methods and process parameters may vary as would be understood by one of ordinary skill in the art. Similarly, the terminology used herein is only for the purpose of describing particular embodiments of the present disclosure and is not intended to limit the scope of the disclosure in any manner.

All publications, patents, and patent applications cited herein, whether supra or infra, are hereby incorporated by reference in their entirety to the same extent as if each individual publication, patent, or patent application was specifically and individually indicated to be incorporated by reference.

The term “comprising” which is synonymous with “including,” “containing,” or “characterized by,” is inclusive or open-ended and does not exclude additional, unrecited elements or method steps.

It will be noted that, as used in this specification and the appended claims, the singular forms “a,” “an” and “the” include plural referents unless the content clearly dictates otherwise. Thus, for example, reference to a “partition” includes one, two, or more partitions.

As used in the specification and appended claims, directional terms, such as “top,” “bottom,” “left,” “right,” “up,” “down,” “upper,” “lower,” “proximal,” “distal” and the like are used herein solely to indicate relative directions and are not otherwise intended to limit the scope of the disclosure or claims.

Where possible, like numbering of elements have been used in various figures. Furthermore, multiple instances of an element and or sub-elements of a parent element may each include separate letters appended to the element number. For example, two instances of a particular element “10” or two alternative embodiments of a particular element may be labeled as “10A” and “10B”. In that case, the element label may be used without an appended letter (e.g., “10”) to generally refer to all instances of the element or any one of the elements. Element labels including an appended letter (e.g., “10A”) can be used to refer to a specific instance of the element or to distinguish or draw attention to multiple uses of the element. Furthermore, an element label with an appended letter can be used to designate an alternative design, structure, function, implementation, and/or embodiment of an element or feature without an appended letter. Likewise, an element label with an appended letter can be used to indicate a sub-element of a parent element. For instance, an element “12” can comprise sub-elements “12A” and “12B.”

Various aspects of the present devices and systems may be illustrated by describing components that are coupled, attached, and/or joined together. As used herein, the terms “coupled”, “attached”, and/or “joined” are used to indicate either a direct connection between two components or, where appropriate, an indirect connection to one another through intervening or intermediate components. In contrast, when a component is referred to as being “directly coupled”, “directly attached”, and/or “directly joined” to another component, there are no intervening elements present. Furthermore, as used herein, the terms “connection,” “in communication,” “connected,” and the like do not necessarily imply direct contact between the two or more elements.

Various aspects of the present devices, systems, and methods may be illustrated with reference to one or more exemplary embodiments. As used herein, the term “embodiment” means “serving as an example, instance, or illustration,” and should not necessarily be construed as preferred or advantageous over other embodiments disclosed herein.

Unless defined otherwise, all technical and scientific terms used herein have the same meaning as commonly understood by one of ordinary skill in the art to which the present disclosure pertains. Although methods and materials similar or equivalent to those described herein can be used in the practice of the present disclosure, the preferred materials and methods are described herein.

The present disclosure relates to bioprocess control systems, bioproduction systems, computing devices, modules, libraries and user interfaces. More specifically, the present disclosure relates to bioprocessing instrument control system modules, libraries, and user interfaces used to control bioprocessing instruments and biological processes. In various embodiments, bioprocessing includes processes that use or yield biological reagents or end-products including, but not limited to, blood, carbohydrate, cell, cell media, drug, enzyme, lipid, nucleic acid (e.g., DNA, RNA), pharmaceutical, plasmid, protein, reagent, vaccine, viral vector and virus reagents and end-products.

In various embodiments, bioprocessing instruments generally include any instrument used to perform all, or part of a bioproduction process that includes a computing device or a component thereof (e.g., processor, memory, firmware) in operation of the instrument, such as, but not limited to, bioreactors, blood processing systems, cell culture systems, cell processing systems, centrifuges, centrifugal separators, electrophoresis systems, electroporation systems, fermenters, fluid management systems and monitors, freezers, imaging systems, flow cytometry systems, sequencing systems, chromatography systems, oligonucleotide amplification or detection systems, incubators, mixers, fill and finish systems, magnetic bead processing systems, and/or other instruments capable of producing, processing, mixing, managing and storing biological reagents and end-products, such as cells and cell products.

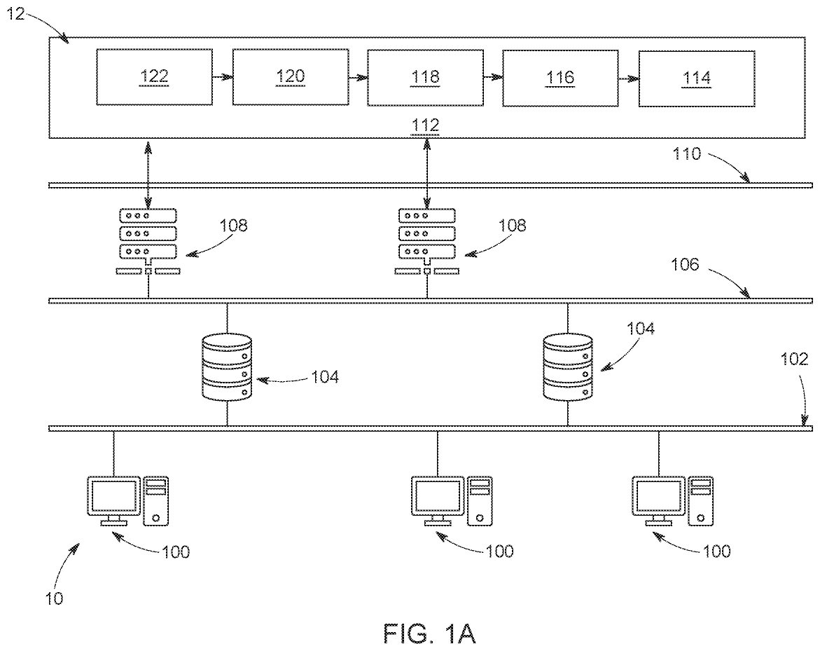

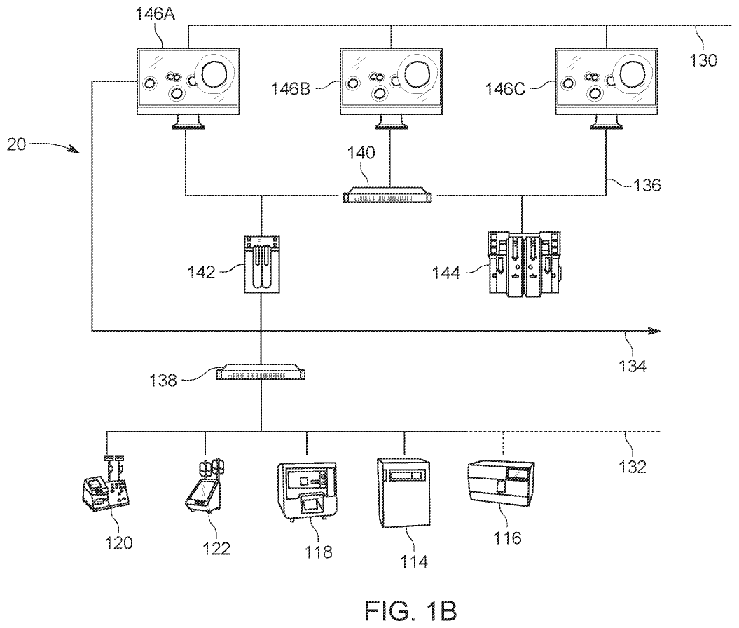

FIG. 1A depicts a block diagram of a bioproduction system 10 in accordance with example embodiments. Example system 10 includes one or more computing devices 100 with user interfaces implemented on a display of the computing device. The computing device 100 can display a graphical user interface(s) that corresponds to one or more specific bioprocessing instrument(s) 114, 116, 118, 120, 122 of the bioprocessing instrument set 112 and configured to receive a virtual input from a user, as well as readable instrument and process parameter outputs (e.g., a device or process state) for controlling operation and/or monitoring of a bioproduction process (e.g., cell therapy manufacturing workflow) performed by a bioprocessing instrument(s) 114, 116, 118, 120, 122, composing a bioprocessing instrument set 112.

In embodiments, the computing device(s) 100 can receive operator inputs through the graphical user interface presented on a display of the computing device, and inputs are communicated to one or more computing devices 104 (e.g., controllers, switches, processors and the like). Communication can be achieved by any wired and/or wireless network including, for example, a wide area network (WAN), a local area network (LAN), a virtual local area network (VLAN), a public land mobile network (PLMN), the Internet, and/or the like. All data interactions including sending, receiving, writing, overwriting, copying instructions between above components, namely computing devices, bioprocessing instruments, and any associated intervening computing devices, network switches, controllers or otherwise, can be stored in memory of any component, e.g., computing device, secondary computing device, computing device of a bioprocessing instrument, network switch and/or controller, and/or remote server (e.g., portable computer, tablet, cell phone, cloud, and the like).

In various embodiments, computing devices 104 include one or more processors for running software and memory for storing data, software, software databases, libraries, and/or modules corresponding to the identity, state, and operation of specific bioprocessing instrument(s) 114, 116, 118, 120, 122 in the bioprocessing instrument set 112. In some embodiments, a plurality of computing devices is utilized, such as a computing device including a display having graphical user interface options for inputting commands to control the bioprocessing instrument set (collectively or independently). Computing devices 104 may also include firmware, software databases, and libraries including software and/or firmware that dictates input/output communication between an instrument and a computing device to transmit operational controls determined by virtual input from a user thereby facilitating operational control of one or more instruments of the bioprocessing instrument set. In embodiments, computing devices 104 include functionality that dictates how the bioprocessing instrument(s) 114, 116, 118, 120, 122 in the bioprocessing instrument set 112 communicate through a field bus 106 (or other communication means) with one or more controllers 108, receive data from one or more controllers 108, transmit data to one or more controllers 108 and generally interface with one or more controllers 108.

One or more controllers 108 can transmit control commands, read and write data over a communication link 110 to the bioprocessing instrument(s) 114, 116, 118, 120, 122 in the bioprocessing instrument set 112 to control the instruments 114, 116, 118, 120, 122 and their operation in a biological process. The bioprocessing instrument(s) 114, 116, 118, 120, 122 can in turn send operational data and other read and write data corresponding to device identity, device state and device communication requirements to one or more controllers 108. In response, the controllers 108 and/or computing devices 104 communicating with controllers 108 transmit and display process parameters and instrument identity, state and operational data at a user interface of a client computing device 100. Controllers 108 and bioprocessing instruments 114, 116, 118, 120, 122 can communicate using one or more communication protocols, including but not limited to, OPC-UA, Modbus, Fieldbus, Profibus and/or others. In an example embodiment, OPC-UA communication protocol is used to collect, store and process read and write data transmitted from bioprocessing instrument(s) 114, 116, 118, 120, 122 to one or more controllers 108 and/or computing devices 104.

In other illustrative embodiments, predefined recipes corresponding to the predetermined operation of bioprocessing instrument(s) 114, 116, 118, 120, 122 can be stored in instrument specific libraries or as instrument specific software modules at one or more computing devices 104 and/or 100. The predefined recipes can be pulled and transmitted to one or more controllers 108 based on an operator input at a user interface of a client computing device 100. In response, one or more controllers 108 can transmit control data to bioprocessing instrument(s) 114, 116, 118, 120, 122 that carry-out a predefined set of operations in accordance with the specific recipe for a biological process. Providing a tailored and streamlined dataset to the controller 108 with instrument specific libraries and software modules stored in memory of computing devices 104 (e.g., located at the bioproduction plant control level) optimizes the field and process level operations, increases the efficiency of the biological process and reduces user error. This is practically relevant in complex biological processes with multiple interconnected instruments performing diverse operations.

For each of the bioprocessing instruments of the bioprocessing instrument set, equipment module detail faceplates show more detailed information of the module. In embodiments, the equipment module faceplates include four tabs which can be used to select different types of parameters including, for example, operator, tuning, display and batch recipe. In embodiments, parameters shown on the operator parameter tab may be modified by a user with operator privileges, and they are used to control how the protocol runs operation of bioprocessing instrument(s) 114, 116, 118, 120, 122 in a bioprocessing instrument set 112. Parameters shown on the tuning parameter tab may be modified only by a user with configuration privileges, and they are used to control how the user interface functions. Parameters shown on the display parameter tab are internally calculated for display only and cannot be modified. Parameters shown on the tuning parameter tab may be modified by a user with operator privileges, and are used to control how a given protocol runs.

In various embodiments, phase classes contained within individual bioprocessing instruments 114, 116, 118, 120, 122 are configured to: 1) initialize the instrument for operational control; 2) issues prompts, wait for a response, and then store the answer; and 3) commands equipment module to start protocol run, waits for equipment module to stop protocol run and stores critical parameters of the bioprocessing instrument(s) 114, 116, 118, 120, 122.

In general, a batch, recipe or batch recipe for use on a bioprocessing instrument is inputted through the graphical user interface and includes a set of instructions given by a user (inputted into a computing device of the disclosure) to carry out operations on a given instrument contained within the bioprocessing instrument set. The creation of batch recipes may include editing, modifying, updating, resetting, and/or creating parameters for various components of a bioprocessing instrument, selecting inlet/outlet paths/manifolds for individual instrument operations. Batch recipes can also include organizing, rearranging, prioritizing, and reprioritizing steps or sub-steps related to operational process of an individual instrument of the system or operational processes between instruments within the bioprocessing instrument set to generate, e.g., a cell therapy product.

In the illustrative embodiment depicted in FIG. 1A, the bioprocessing instrument set 112 forms an interconnected cell processing system 12, including a blood processing system 122, a magnetic bead processing system 120, an electroporation system 118, a freezing system 116 and an incubator system 114. The interconnected cell processing system 12 forms a part of the bioprocessing system 10. In embodiments, the bioprocessing instrument set 112 and interconnected cell processing system 12 further includes a fill and finish system.

In illustrative embodiments, the blood processing system 122 can include a fluid management kit with flow paths controlling the flow of fluid or cells through the kit, a pump, and a centrifugation system that can perform a broad range of cell processing applications, such as separation and isolation of cell types for Chimeric antigen receptor (CAR)-T therapy, stem cell therapy, and peripheral blood mononuclear cells (PBMCs) isolation. For example, the blood processing system 122 can be the CTS Rotea™ Counterflow Centrifugation System provided by Thermo Fisher Scientific Inc. Additional blood processing systems 122 and associated features and components are disclosed in WO 2018/204992, which is hereby incorporated by reference in its entirety herein. The blood processing system 122 can consist of a compact multipurpose instrument and a sterile single-use kit. Together these components constitute a high throughput closed system that offers exceptional cell recovery, and flexible input and output volume capability. The example blood processing systems 122 disclosed herein can perform leukopak processing, including isolation and extraction of monocytes, lymphocytes, red blood cells, platelets, and plasma from a leukopak. In an exemplary embodiment, the blood processing system 122 is used to extract, separate and/or isolate T-cells in a leukopak.

In illustrative embodiments, the magnetic bead processing system 120 can attach magnetic beads and ligands to the output and end-product of the blood processing system 122. For example, the magnetic bead processing system 120 can be the bead processing system described in PCT/US2021/054490, which is hereby incorporated by reference in its entirety herein. In an example embodiment, the magnetic bead processing system 120 can attach magnetic beads and/or ligands to T-cells, activate T-cells and harvest the activated T-cells for further processing as described in PCT/US2021/054490.

In illustrative embodiments, the electroporation system 118 can be used to apply an electrical field to the output or end-product of the magnetic bead processing system 120 in order to increase the permeability of the cell membrane, allowing chemicals, drugs, electrode arrays, RNA or DNA to be introduced into the cell. In an exemplary embodiment, the electroporation system 118 is used to introduce a payload, such as a protein or oligonucleotide, such as RNA or DNA into a T-cell or NK-cell. As an illustrative example, the electroporation system 118 can be the electroporation system described in PCT/US2020/057138, which is hereby incorporated by reference in its entirety herein.

In illustrative embodiments, the freezing system 116 can be used to freeze and preserve the output or end-product of the electroporation system 118. For example, the freezing system 116 can be the CryoMed™ Controlled-Rate Freezer provided by Thermo Fisher Scientific Inc. The CryoMed™ Controlled-Rate Freezer (CRF) provides precise, repeatable freezing results that protect samples from intracellular freezing. In an example embodiment, T-cells impregnated with RNA or DNA can be frozen and preserved using the freezing system 116.

In various embodiments, the incubator may be used to incubate the product or input of any of the bioprocessing instruments. In some embodiments, the incubator system 114 can be used to incubate the output or end-product of the freezing system 116. In some embodiments, the incubator system 114 is used to incubate genetically modified cells generated via the electroporation system 116. In various embodiments, the incubator system 114 is used to expand cells, such as genetically modified cells generated via the electroporation system 116. In some embodiments, the incubator system 114 is used to incubate T-cells impregnated with a gene editing reagent including enzymes, RNA, DNA or various combinations thereof.

The bioprocessing instrument set 112 can include any number and/or combination of other bioprocessing instruments. For example, in some embodiments, the bioprocessing instrument set 112 can include a fill and finish system for formulation and dispensing into dosage format of cellular therapeutic products produced by the system of the disclosure. In some embodiments, the fill and finish system is a type disclosed in WO2023/272360, incorporated herein by reference in its entirety. In embodiments shown in WO2023/272360, the fill and finish system, also referred to as a liquid handling system, is configured for preparation of small volume liquid formulations and dispensing into output vessels before use or freezing. The system can be utilized for a variety of low volume liquid formulation preparation applications (e.g., addition of a cryoprotectant media to a cell containing sample), with cell therapies being one example application.

In other examples, the bioprocessing instrument set 112 includes an autologous cell therapy system and process flow, including one or more bioreactors or bioreactor pods operating in static and/or dynamic modes. In some embodiments, the system is a type disclosed in PCT/US2023/074211, incorporated herein by reference in its entirety. In embodiments shown in PCT/US2023/074211, the autologous cell therapy system includes one or more equipment modules, including blood processing systems 122, cell and bead processing systems 120, electroporation systems 118, bioreactors/bioreactor pods (e.g., bioreactor pod and/or bioreactor), incubators 114, freezers 116 and associated automation software for controlling each equipment module with their respective controller.

In example embodiments, the autologous cell therapy system can include three blood processing systems 122 and two bioreactors/pods, one electroporation system 118, one incubator 114 and one freezer 116. The three blood processing systems 122 and two bioreactors/pods can be the same systems/bioreactors where cells and media are flowed and recycled or separate systems/bioreactors. Any combination of equipment modules described herein can be combined and customized to meet the operator and patient's needs. In other example embodiments, the autologous cell therapy system can include three blood processing systems 122 and two bioreactors/pods, one cell and bead processing system 120, one electroporation system 118, and one freezer 116. The bioreactor/pod can operate as an incubator with the use of one or more top or bottom spargers, and therefore, incubator 114 is not necessary if a dual-mode bioreactor is implemented.

In various embodiments, the bioprocessing instrument(s) 114, 116, 118, 120, 122 in the bioprocessing instrument set 112 can all be controlled independently using a single, or multiple graphical user interfaces provided at one or more computing devices 100 to one or more operators.

FIG. 1B depicts a hardware architecture 20 for bioprocess control system (BPCS) 10 in accordance with example embodiments. The architecture 20 can include a plurality of inter-network layers extending between bioprocessing instrument(s) 114, 116, 118, 120, 122 in the bioprocessing instrument set 112 and a base network 130. For example, as shown in FIG. 1B, the plurality of layers can include a first OPC UA network layer 132, a second OPC UA network layer 134, and a distributed control system (DCS; for example, DeltaV™ Area Control Network (I)) 136.

The first OPC UA network layer 132 facilitates communication between OPC servers connected to applications operating on bioprocessing instrument(s) 114, 116, 118, 120, 122 in the bioprocessing instrument set 112 and the second OPC UA network layer 134 via a first network switch 138. For example, the first OPC UA network layer 132 is configured to connect with each of the Dynacellect™ Magnetic separation System, Rotea™ Counterflow Centrifugation System, Xenon™ Electroporation System, Heracell™ VIOS CO2 Incubator, and CryoMed™ Controlled Rate Freezer.

The second OPC UA network layer 134 facilitates communication between the first OPC UA network 132 and the DCS 136 via a second network switch 140. Additionally, an Ethernet I/O Card (EIOC) 142 and PK controller 144 can be connected to the DCS. EIOC 142 provides a platform to access data from Ethernet devices in the DCS. Ethernet Devices capable of talking Modbus TCP, EtherNet/IP, IEC 61850 MMS (Manufacturing Message Specification), OPC UA client and EtherNet/IP Control Tag Integration protocols are supported. The EIOC provides monitoring and control of Ethernet Devices on the Ethernet Device Network via control modules assigned to and executed in the EIOC. Ethernet Devices like PLCs, Motor Control Centers, drives, switchgear and others can be controlled directly by the EIOC, independent of a controller. PK controller 144 provides for seamless merging into a DCS system resulting in unified database and system for operation of various components in software architecture 20.

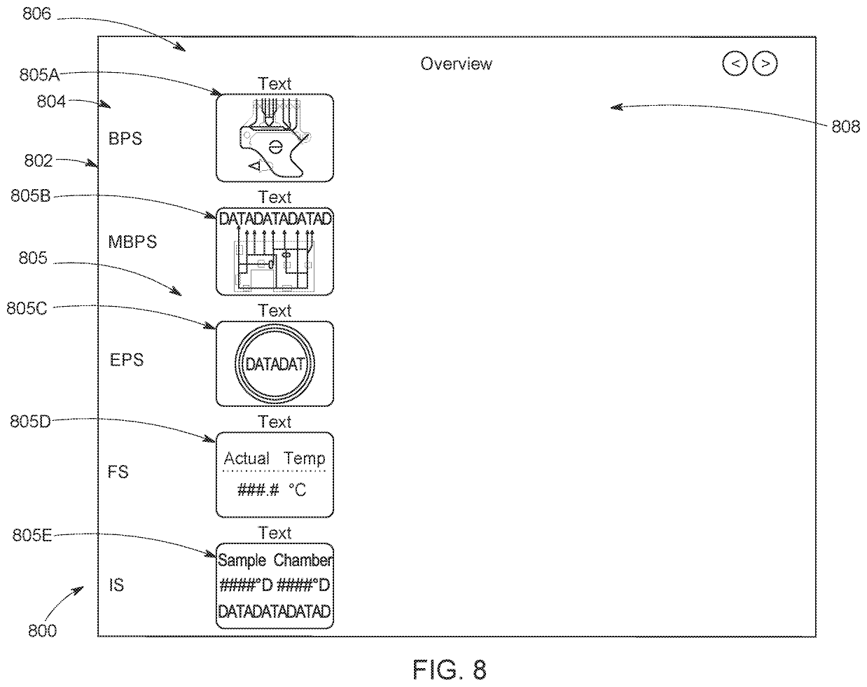

The DCS 136 facilitates communication between the second OPC UA network layer 134 and the base network 130 via an app station OPC client 146A, Operator station 146B, and a ProPlus module 146C. The operator station 146B includes a display for displaying the graphical user interface(s) to facilitate operational control of bioprocessing instrument(s) and provide the user with a virtual output indicating a process state or a device state of a given bioprocessing instrument as depicted in FIGS. 8-14. In embodiments, the operator station 146B is a computing device which includes functionality for chronicling and displaying alarms and event status of a bioprocessing instrument, as well as provide historian functions for an instrument. In embodiments, the ProPlus module 146C is a computing device which includes functionality for chronicling and displaying alarms and event status of a bioprocessing instrument and performs batch executive functions and batch and continuous historian functions. App station 146A is a computing device which includes a processor in connection with a memory storing one or more software modules configured for communicating with and controlling operation of a bioprocessing instrument.

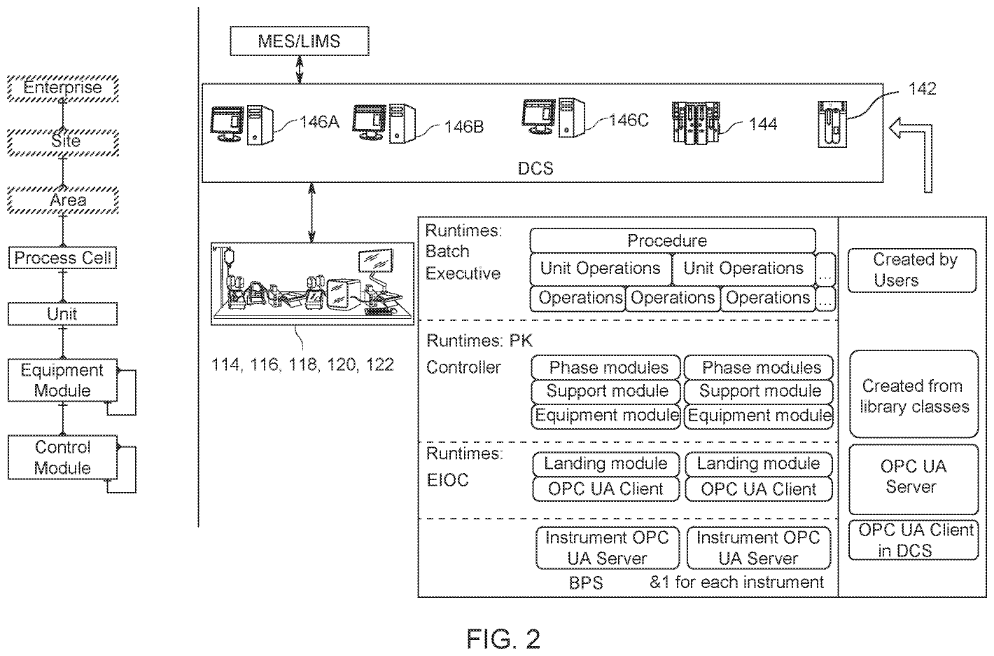

FIG. 2 is a workflow diagram for DCS system in various embodiments for controlling and monitoring bioprocess system 10. In illustrative embodiments DCS system can be employed at a manufacturing plant including, enterprise, site, area, process cell, unit, equipment module, and control module features. Manufacturing execution software system (MES)/Lab information management software system (LIMS) can be in electronic communication with a distributed control system (DCS) for controlling and monitoring operations of bioprocess instrument set 12 shown in FIG. 1. DCS system includes various components including computing units 146, 146B, 146C, controllers 144 and Ethernet I/O Card (EIOC) 142. DCS system controls, monitors, and facilitates operations of instruments in bioprocess system set including blood processing system 122, magnetic bead processing system 120, electroporation system 118, freezer system 116, and incubator system 114 through batch operations including unit operations, phase module, support module, equipment module, landing module, and OPC UA server.

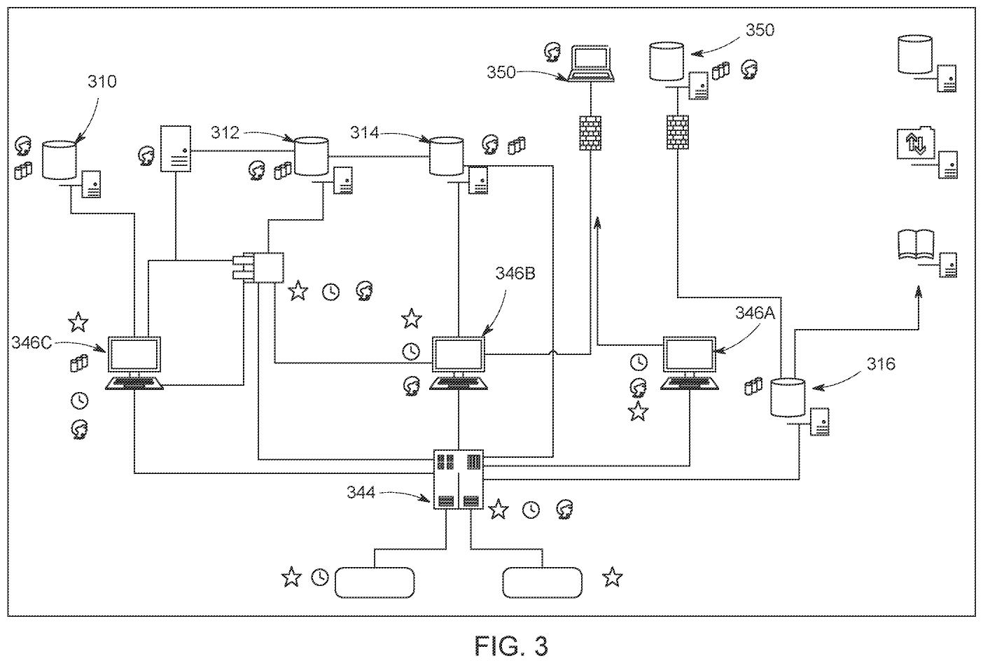

FIG. 3 is an illustrative flow diagram of the DCS system in various embodiments. To meet GMP standard for producing a cell based therapeutic, information of the workflow must be maintained and recorded. FIG. 3 depicts a system of the disclosure including a multitude of computing devices, intermittent controllers and network switch overlays. In embodiments, as illustrated in FIG. 3, the DCS architecture includes a plurality of inter-network layers extending between the bioprocessing instrument in the bioprocessing instrument set (not shown) and a base network. For example, the plurality of layers may include a first network layer, a second network layer, and a DCS.

The first network layer is operable to facilitate communication between an OPC server connected to an application operating on the bioprocessing instrument and the second network layer via a first network switch. The second network layer facilitates communication between the first network and the DCS via a second network switch. Additionally, an EIOC and controller are communicatively connected to the DCS. EIOC provides a platform to access data from Ethernet devices in the DCS. Controller provides for seamless merging into the DCS resulting in a unified database and system for operation of various components in the architecture.

The DCS facilitates communication between the second network layer and the base network via an app station OPC client 346A, Operator station 346B, and a ProPlus module 346C. The operator station 346B includes a display for displaying the graphical user interface(s) to facilitate operational control of bioprocessing instrument(s) and provide the user with a virtual output indicating a process state or a device state of a given bioprocessing instrument as depicted in FIGS. 8-14. In embodiments, the operator station 346B is a computing device which includes functionality for chronicling and displaying alarms and event status of a bioprocessing instrument, as well as provide historian functions for an instrument. In embodiments, the ProPlus module 346C is a computing device which includes functionality for chronicling and displaying alarms and event status of a bioprocessing instrument and performs batch executive functions and batch and continuous historian functions. App station 346A is a computing device which includes a processor in connection with a memory storing one or more software modules configured for communicating with and controlling operation of a bioprocessing instrument.

In embodiments, the system includes controller 344 which is communicatively connected to DCS and various computing devices (310, 312, 314, 316) having functionality for one or more of system security, batch historian functions, storage, even chronicling, web services, digital record signature and storage, data integrity functionality, batch monitoring, wherein one or more of the computing device may be communicatively connected to a remote computing device 350 (such as a server, cloud, remote terminal and the like).

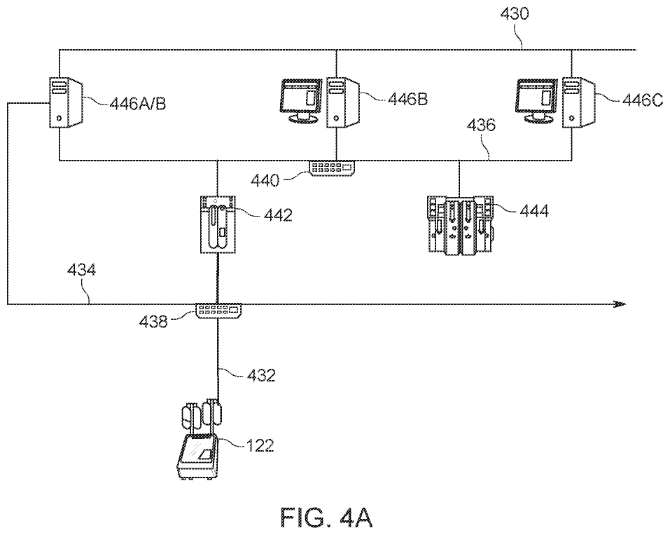

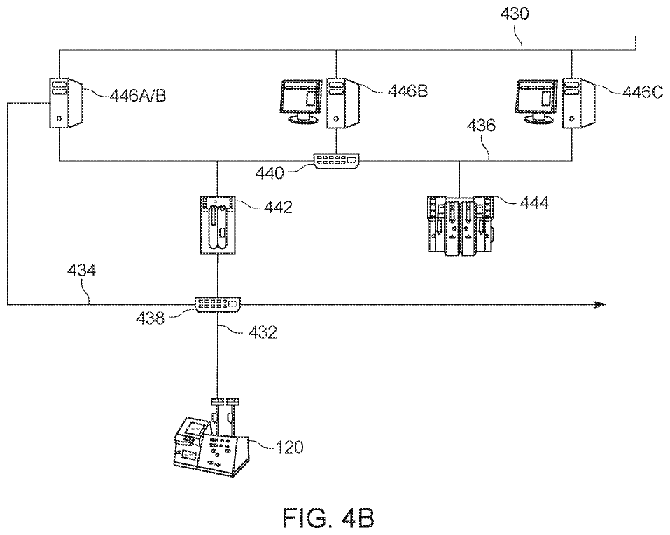

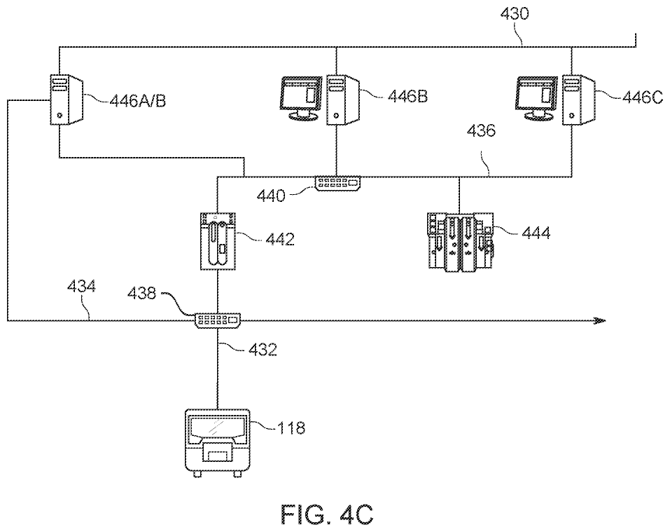

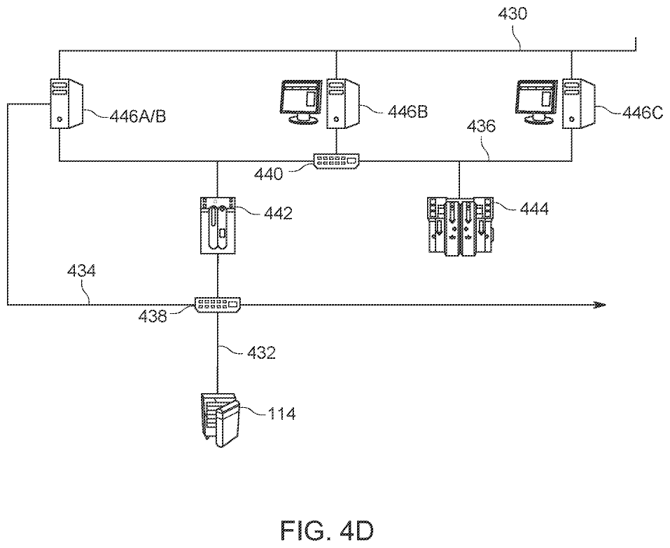

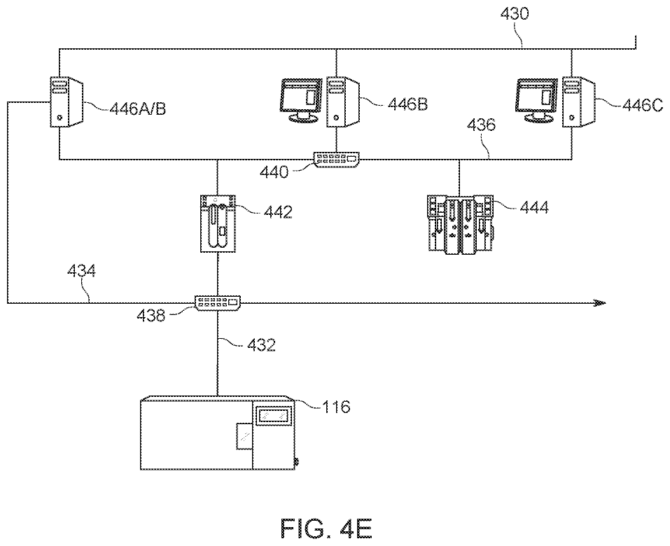

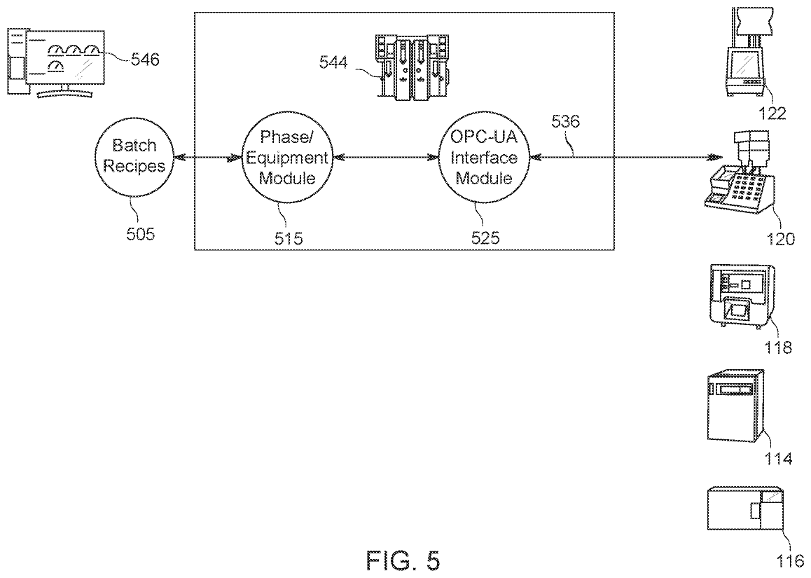

FIGS. 4A-4E depict hardware architectures for showing system connectivity to a single bioprocessing instrument of the bioprocessing instrument set. FIG. 4A depicts a hardware architecture for a bioprocess control/bioproduction system including a blood processing system 122, such as a Rotea™ Counterflow Centrifugation System. In embodiments, the architecture includes a plurality of inter-network layers extending between the bioprocessing instrument 122 in the bioprocessing instrument set and a base network 430. For example, as shown in FIG. 4A, the plurality of layers includes a first network layer 432, a second network layer 434, and a DCS 436.

The first network layer 432 is operable to facilitate communication between an OPC server connected to an application operating on the blood processing system 122 and the second network layer 434 via a first network switch 438. For example, the first network layer 432 is configured to connect with the blood processing system 122 (e.g., Rotea™).

The second network layer 434 facilitates communication between the first network 432 and the DCS 436 via a second network switch 440. Additionally, an EIOC 442 and controller 444 are communicatively connected to the DCS. EIOC 442 provides a platform to access data from Ethernet devices in the DCS. Controller 444 provides for seamless merging into the DCS 436 resulting in a unified database and system for operation of various components in the architecture.

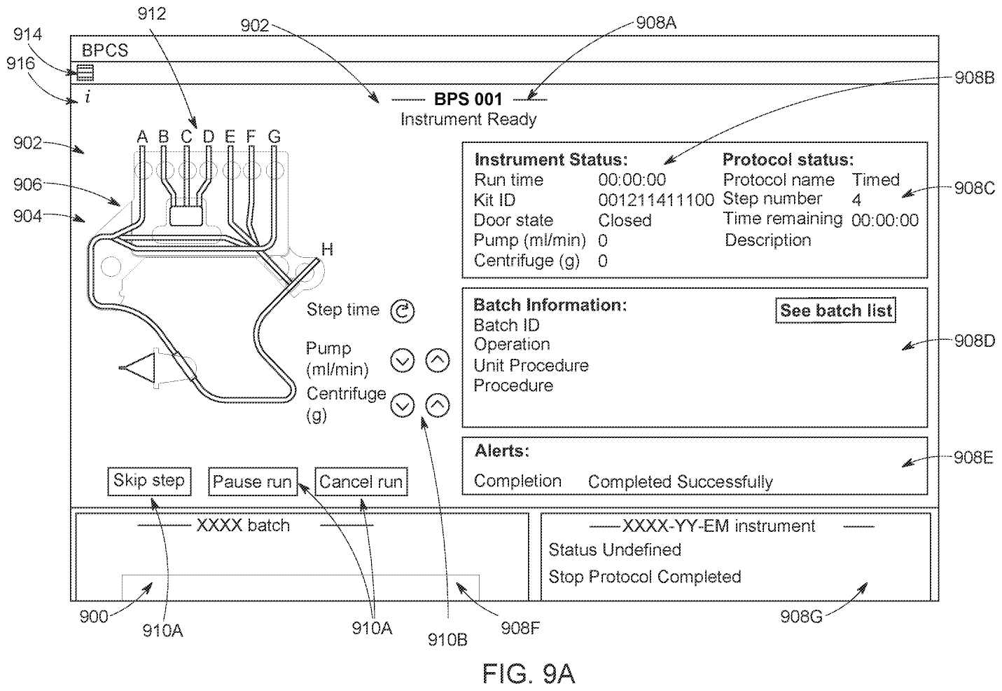

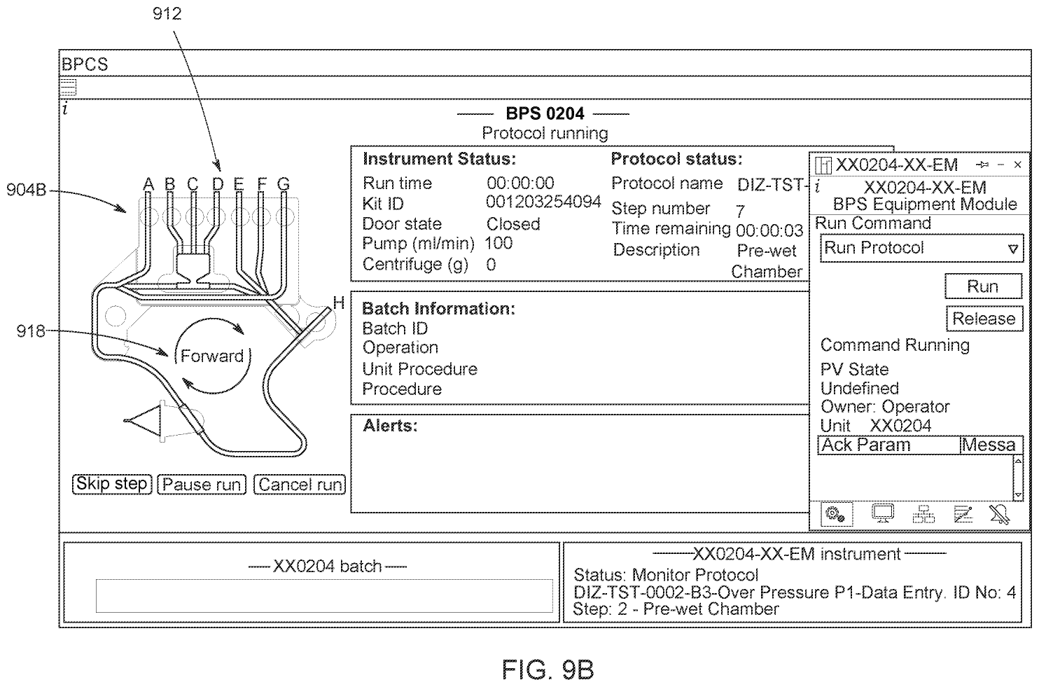

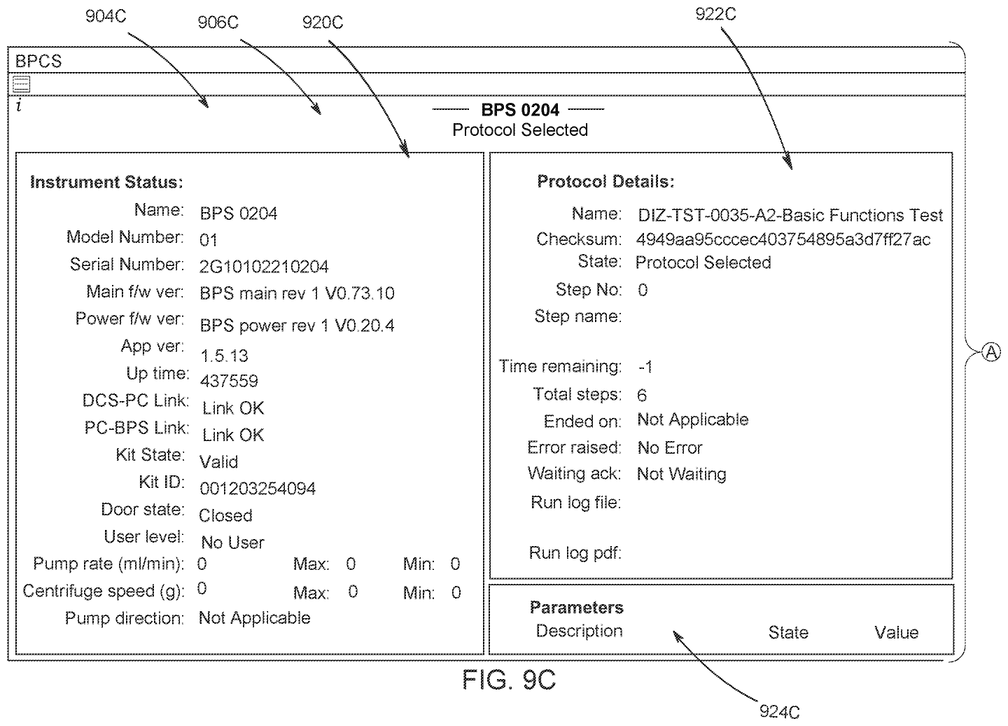

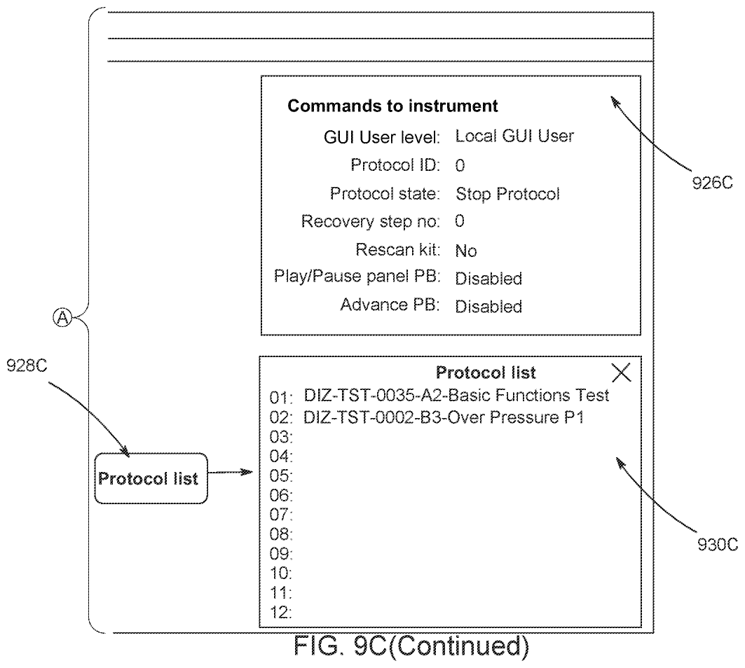

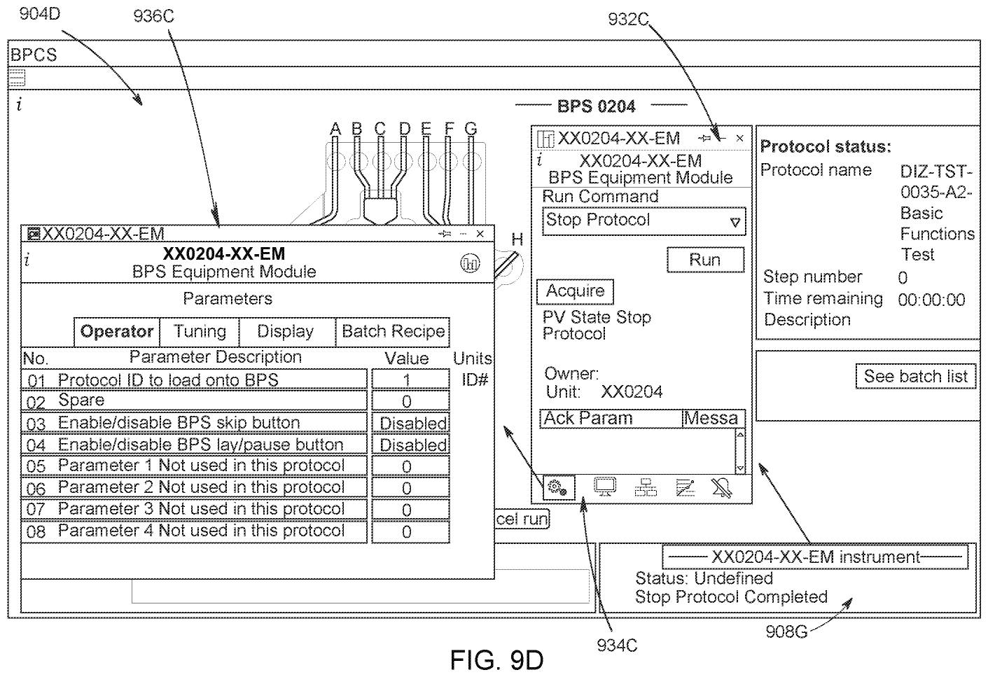

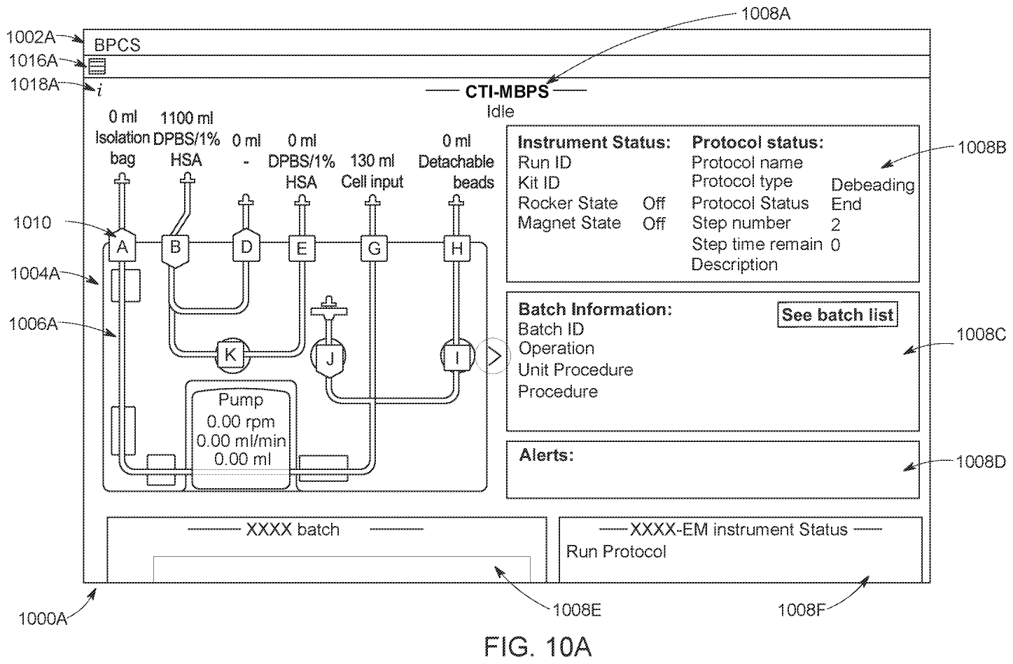

The DCS 436 facilitates communication between the second network layer 434 and the base network 430 via a first computing device 446B, optionally a second computing device 446A, and optionally a third computing device 446C. The first computing device 446B includes a display for displaying a graphical user interface to facilitate operational control of the blood processing system and provide the user with a virtual output indicating a process state and/or a device state of the blood processing system as depicted in FIGS. 9A-9D. Additional functionality to facilitate operational control of the blood processing system provided in the graphical user interface allows a user to stop/Run Protocol/Run Recovery from Step/Run Recovery Single Step/Monitor Protocol/Recovery Control protocol run. In various embodiments, the additional functionality to facilitate operational control of the blood processing system is provided in the graphical user interface to allow a user to stop and Run Protocols. If errors are encountered the graphical user interface allows a user to initiate Run Recovery from Step/Run Recovery Single Step protocols. Non selectable commands that are initiated as a protocol progresses are Monitor Protocol/Recovery Control.

In embodiments, the second computing device 446B includes functionality for chronicling and displaying alarms and event status of the blood processing system, as well as providing historian functions for the blood processing system. In embodiments, the third computing device 446C includes functionality for chronicling and displaying alarms and event status of the blood processing system and performs batch executive functions and batch and continuous historian functions. The second computing device 446A includes a processor in connection with a memory storing one or more software modules configured for communicating with and controlling operation of the blood processing system.

FIG. 4B depicts a hardware architecture for a bioprocess control/bioproduction system including a magnetic bead processing system 120, such as a DynaCellect™—Magnetic Separation System. In embodiments, the architecture includes a plurality of inter-network layers extending between the bioprocessing instrument 120 in the bioprocessing instrument set and a base network 430. For example, as shown in FIG. 4B, the plurality of layers includes a first network layer 432, a second network layer 434, and a DCS 436.

The first network layer 432 is operable to facilitate communication between an OPC server connected to an application operating on the magnetic bead processing system 120 and the second network layer 434 via a first network switch 438. For example, the first network layer 432 is configured to connect with magnetic bead processing system 120 (e.g., DynaCellect™).

The second network layer 434 facilitates communication between the first network 432 and the DCS 436 via a second network switch 440. Additionally, an EIOC 442 and controller 444 are communicatively connected to the DCS. EIOC 442 provides a platform to access data from Ethernet devices in the DCS. Controller 444 provides for seamless merging into the DCS 436 resulting in a unified database and system for operation of various components in the architecture.

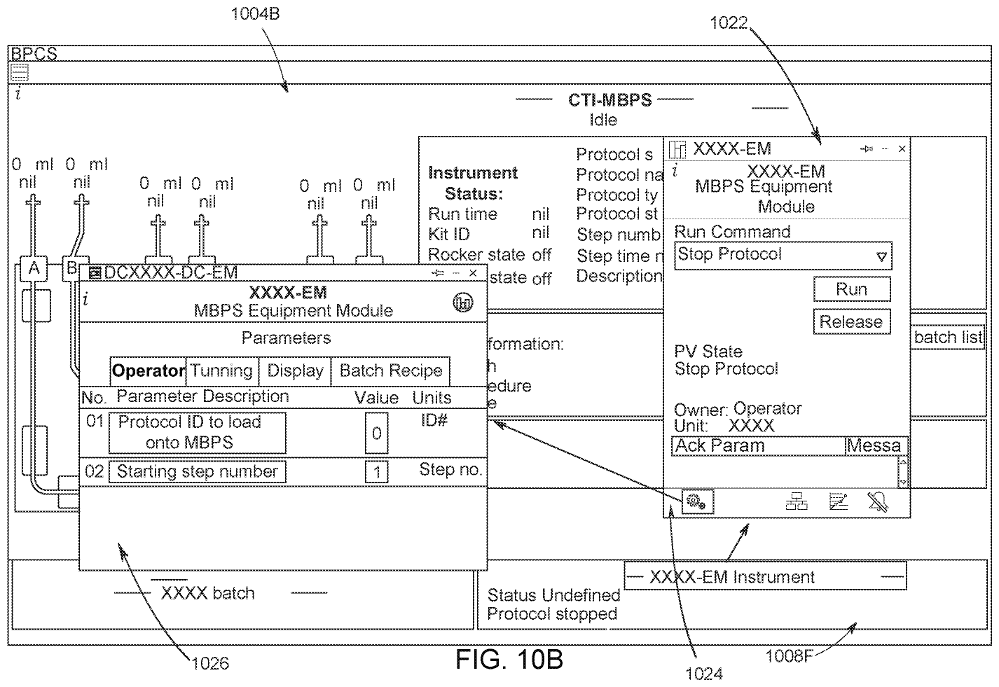

The DCS 436 facilitates communication between the second network layer 434 and the base network 430 via a first computing device 446B, optionally a second computing device 446A, and optionally a third computing device 446C. The first computing device 446B includes a display for displaying a graphical user interface to facilitate operational control of the magnetic bead processing system and provide the user with a virtual output indicating a process state and/or a device state of the magnetic bead processing system as depicted in FIGS. 10A-10B. Additional functionality to facilitate operational control of the magnetic bead processing system provided in the graphical user interface allows a user to Stop protocol/Run Protocol/Run Recovery/Monitor Protocol/Recovery Control protocol run. In some embodiments, additional functionality is provided to facilitate operational control of the magnetic bead processing system in the graphical user interface allowing a user to Stop protocol/Run Protocol. If errors are encountered the graphical user interface allows a user to initiate Run Recovery protocol. Non selectable commands that are initiated as a protocol progresses are Monitor Protocol/Recovery Control.

In embodiments, the second computing device 446B includes functionality for chronicling and displaying alarms and event status of the magnetic bead processing system, as well as providing historian functions for the magnetic bead processing system. In embodiments, the third computing device 446C includes functionality for chronicling and displaying alarms and event status of the blood processing system and performs batch executive functions and batch and continuous historian functions. The second computing device 446A includes a processor in connection with a memory storing one or more software modules configured for communicating with and controlling operation of the magnetic bead processing system.

FIG. 4C depicts a hardware architecture for a bioprocess control/bioproduction system including an electroporation system 118, such as a Xenon™ Electroporation System. In embodiments, the architecture includes a plurality of inter-network layers extending between the bioprocessing instrument 118 in the bioprocessing instrument set and a base network 430. For example, as shown in FIG. 4C, the plurality of layers includes a first network layer 432, a second network layer 434, and a DCS 436.

The first network layer 432 is operable to facilitate communication between an OPC server connected to an application operating on the electroporation system 118 and the second network layer 434 via a first network switch 438. For example, the first network layer 432 is configured to connect with electroporation system 118 (e.g., Xenon™).

The second network layer 434 facilitates communication between the first network 432 and the DCS 436 via a second network switch 440. Additionally, an EIOC 442 and controller 444 are communicatively connected to the DCS. EIOC 442 provides a platform to access data from Ethernet devices in the DCS. Controller 444 provides for seamless merging into the DCS 436 resulting in a unified database and system for operation of various components in the architecture.

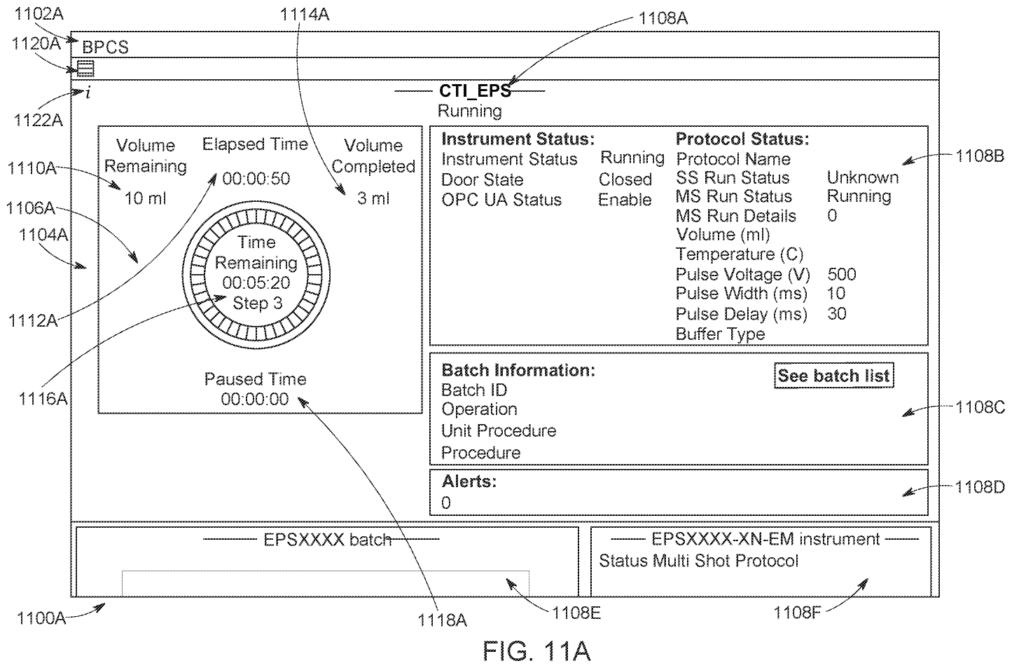

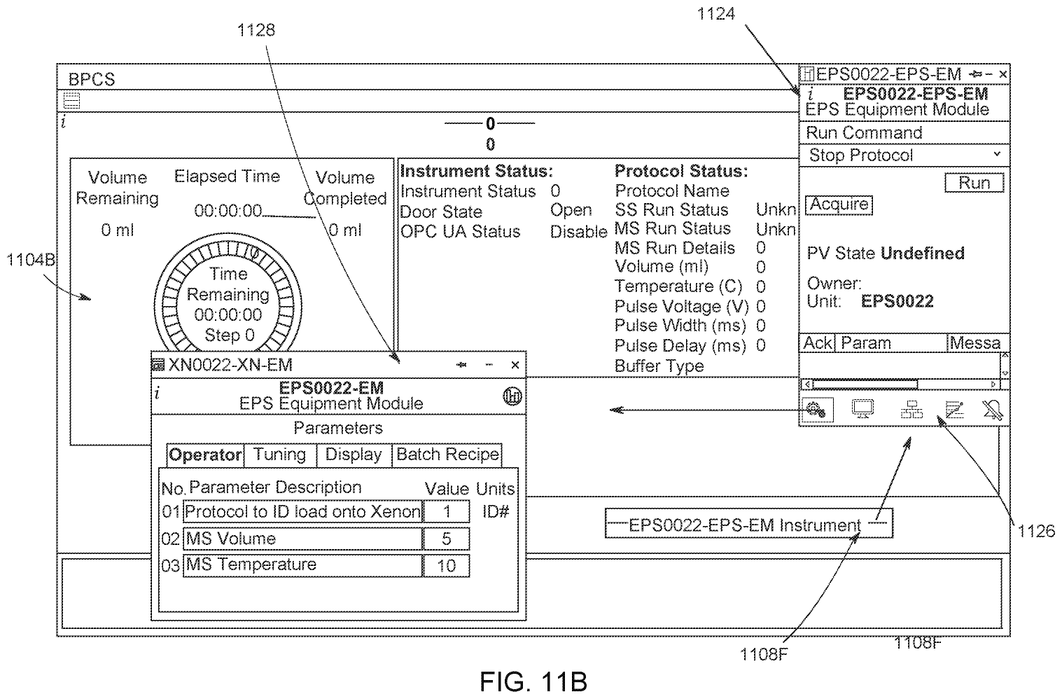

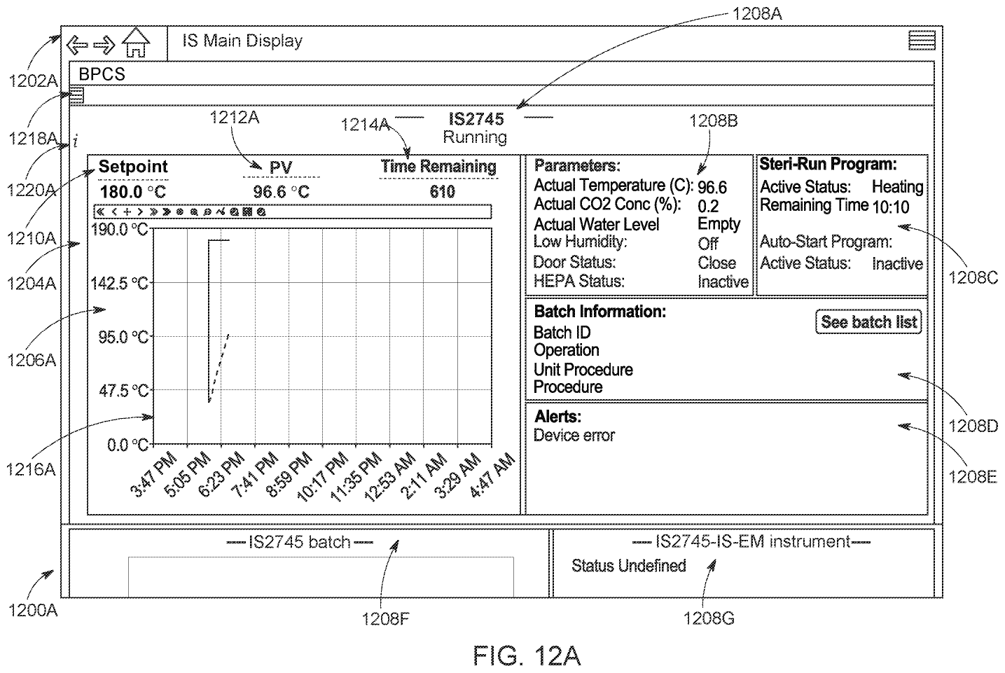

The DCS 436 facilitates communication between the second network layer 434 and the base network 430 via a first computing device 446B, optionally a second computing device 446A, and optionally a third computing device 446C. The first computing device 446B includes a display for displaying a graphical user interface to facilitate operational control of the electroporation system and provide the user with a virtual output indicating a process state and/or a device state of the electroporation system as depicted in FIGS. 11A-11B. Additional functionality to facilitate operational control of the electroporation system provided in the graphical user interface allows a user to Stop Protocol/Single Shot Protocol/Multi Shot Protocol/Monitor Single Shot/Monitor Extraction/Monitor Multi Shot protocol. In embodiments, additional functionality is provided to facilitate operational control of the electroporation system in the graphical user interface allows a user to Stop Protocol/Single Shot Protocol/Multi Shot Protocol. Non selectable commands that are initiated as a protocol progresses are Monitor Single Shot/Monitor Extraction/Monitor Multi Shot protocol.

In embodiments, the second computing device 446B includes functionality for chronicling and displaying alarms and event status of the electroporation system, as well as providing historian functions for the electroporation system. In embodiments, the third computing device 446C includes functionality for chronicling and displaying alarms and event status of the electroporation system and performs batch executive functions and batch and continuous historian functions. The second computing device 446A includes a processor in connection with a memory storing one or more software modules configured for communicating with and controlling operation of the electroporation system.

FIG. 4D depicts a hardware architecture for a bioprocess control/bioproduction system including an incubator system 114, such as Heracell™ VIOS CO2 Incubator. In embodiments, the architecture includes a plurality of inter-network layers extending between the bioprocessing instrument 114 in the bioprocessing instrument set and a base network 430. For example, as shown in FIG. 4D, the plurality of layers includes a first network layer 432, a second network layer 434, and a DCS 436.

The first network layer 432 is operable to facilitate communication between an OPC server connected to an application operating on the incubator system 114 and the second network layer 434 via a first network switch 438. For example, the first network layer 432 is configured to connect with incubator system 114 (e.g., Heracell™ VIOS).

The second network layer 434 facilitates communication between the first network 432 and the DCS 436 via a second network switch 440. Additionally, an EIOC 442 and controller 444 are communicatively connected to the DCS. EIOC 442 provides a platform to access data from Ethernet devices in the DCS. Controller 444 provides for seamless merging into the DCS 436 resulting in a unified database and system for operation of various components in the architecture.

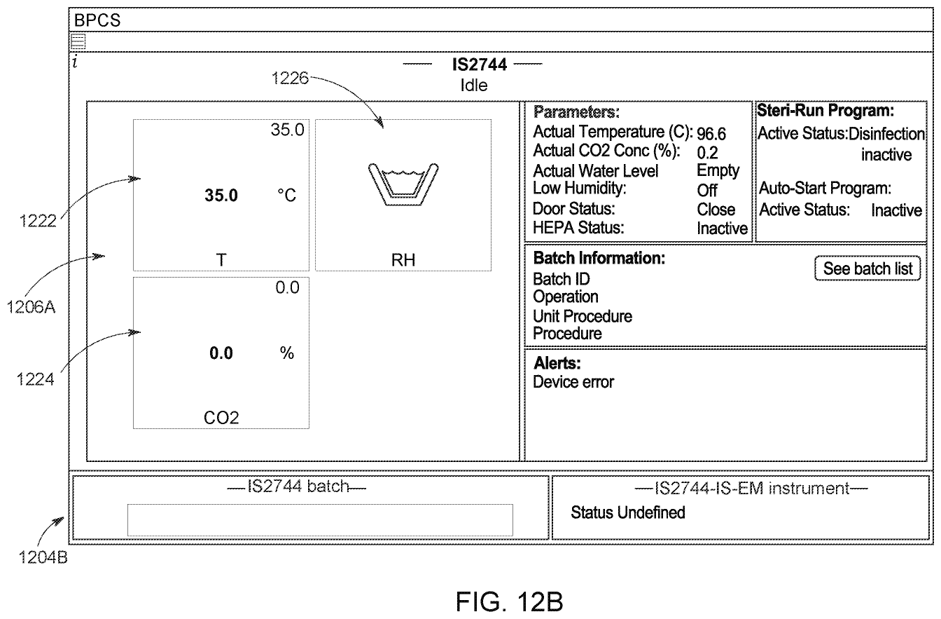

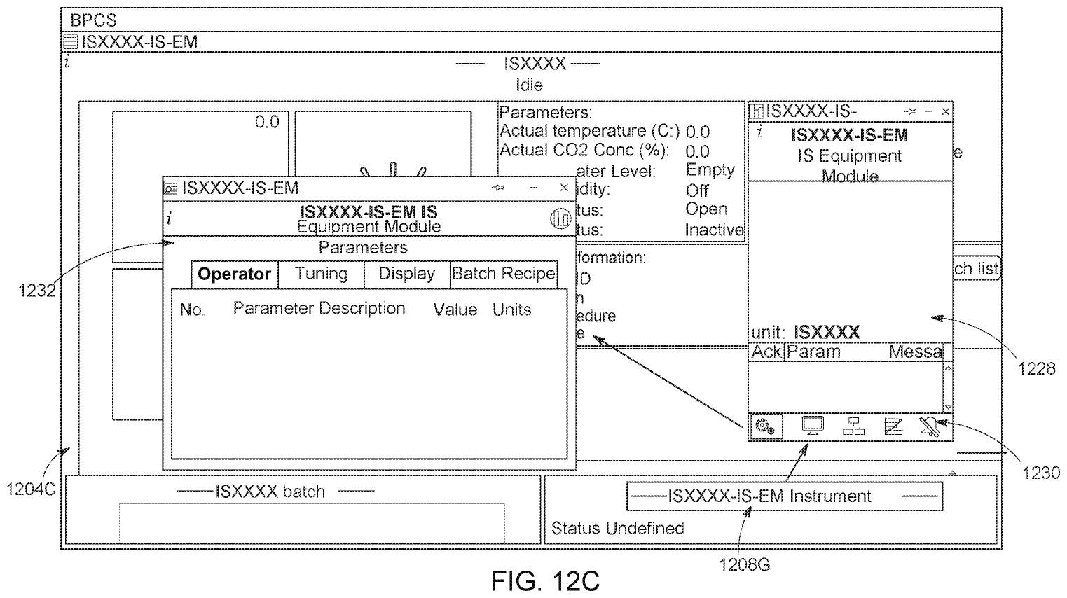

The DCS 436 facilitates communication between the second network layer 434 and the base network 430 via a first computing device 446B, optionally a second computing device 446A, and optionally a third computing device 446C. The first computing device 446B includes a display for displaying a graphical user interface to facilitate operational monitor and/or control of the incubator system and provide the user with a virtual output indicating a process state and/or a device state of the incubator system as depicted in FIGS. 12A-12C.

In embodiments, the second computing device 446B includes functionality for chronicling and displaying alarms and event status of the incubator system, as well as providing historian functions for the incubator system. In embodiments, the third computing device 446C includes functionality for chronicling and displaying alarms and event status of the incubator system and performs batch executive functions and batch and continuous historian functions. The second computing device 446A includes a processor in connection with a memory storing one or more software modules configured for monitoring and/or controlling operation of the incubator system.

FIG. 4E depicts a hardware architecture for a bioprocess control/bioproduction system including a freezing system 116, such as CryoMed™ Controlled-Rate Freezer. In embodiments, the architecture includes a plurality of inter-network layers extending between the bioprocessing instrument 116 in the bioprocessing instrument set and a base network 430. For example, as shown in FIG. 4E, the plurality of layers includes a first network layer 432, a second network layer 434, and a DCS 436.

The first network layer 432 is operable to facilitate communication between an OPC server connected to an application operating on the freezing system 116 and the second network layer 434 via a first network switch 438. For example, the first network layer 432 is configured to connect with freezing system 116 (e.g., CryoMed™).

The second network layer 434 facilitates communication between the first network 432 and the DCS 436 via a second network switch 440. Additionally, an EIOC 442 and controller 444 are communicatively connected to the DCS. EIOC 442 provides a platform to access data from Ethernet devices in the DCS. Controller 444 provides for seamless merging into the DCS 436 resulting in a unified database and system for operation of various components in the architecture.

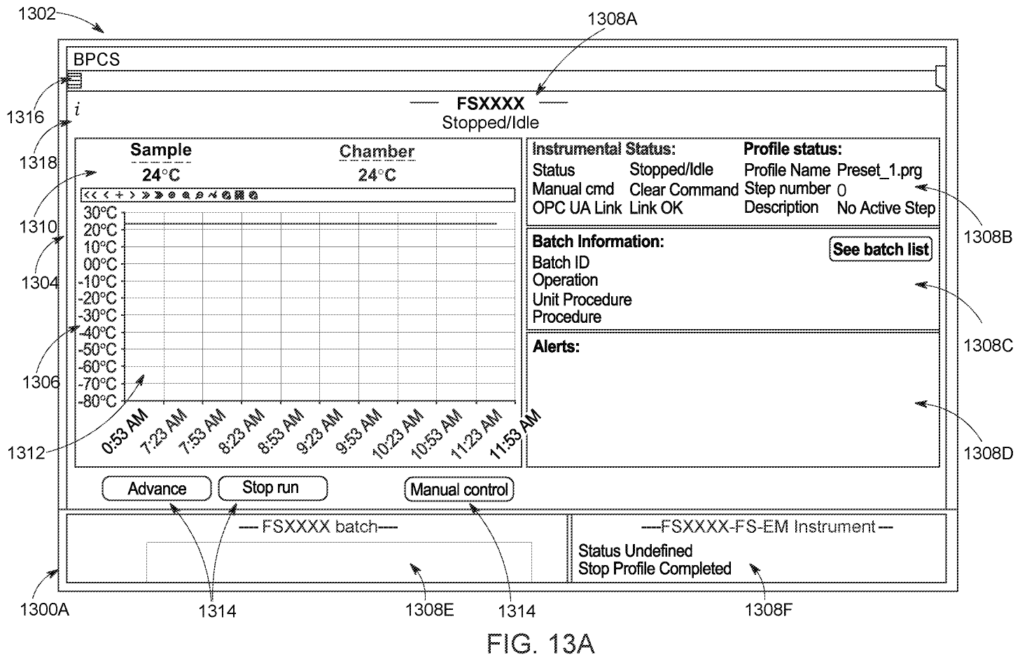

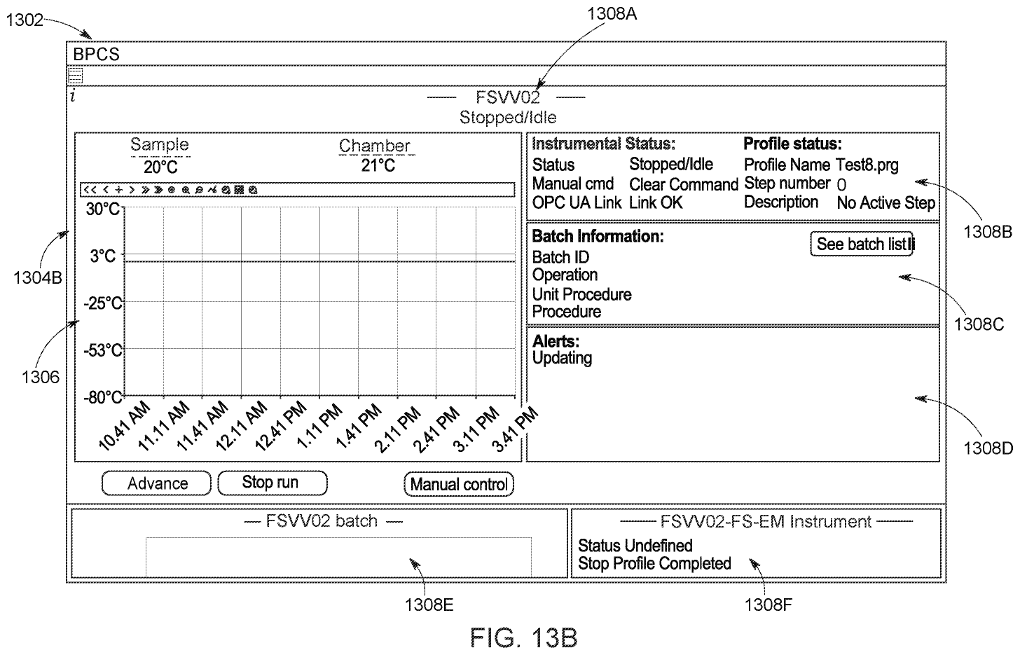

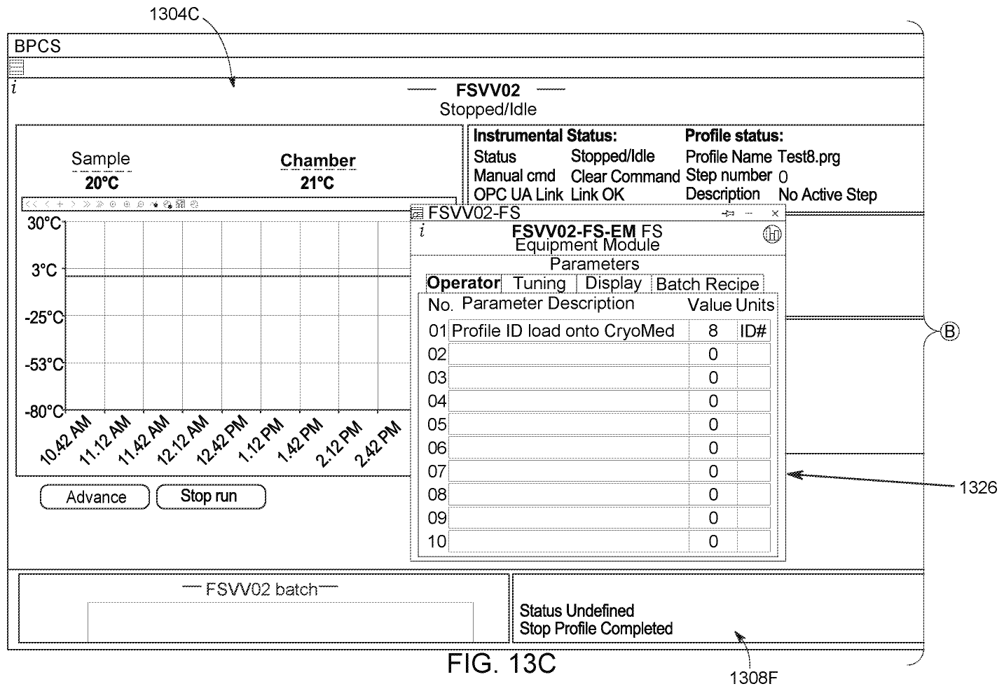

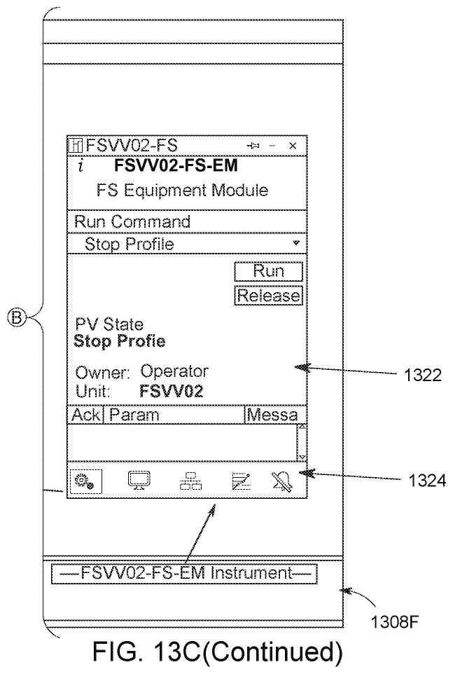

The DCS 436 facilitates communication between the second network layer 434 and the base network 430 via a first computing device 446B, optionally a second computing device 446A, and optionally a third computing device 446C. The first computing device 446B includes a display for displaying a graphical user interface to facilitate operational control of the freezing system and provide the user with a virtual output indicating a process state and/or a device state of the freezing system 116 as depicted in FIGS. 13A-13C.

In embodiments, the second computing device 446B includes functionality for chronicling and displaying alarms and event status of the freezing system 116, as well as providing historian functions for the freezing system. In embodiments, the third computing device 446C includes functionality for chronicling and displaying alarms and event status of the incubator system and performs batch executive functions and batch and continuous historian functions. The second computing device 446A includes a processor in connection with a memory storing one or more software modules configured for communicating with and controlling operation of the freezing system 116.

FIG. 5 is an illustrative batch control and data flow diagram of the DCS system in various embodiments. In general computing system 546 facilitates execution of batch recipes 505. Batch recipes 505 are used to create workflows for instruments including blood processing device 122, magnetic bead processing device 120, electroporation system 118, freezer system 116, and incubator system 114. Each batch and each batch recipe in batch recipes 505 has a unique ID. This allows for all instrument data, including alarms data, events data, historical data to be collated. Batch reports are created via a separate batch reports server based on collected and combined set of instrument data, including alarms data, events data, historical data for each of the instruments including blood processing device 122, magnetic bead processing device 120, electroporation system 118, freezer system 116, and incubator system 114.

Controllers 544 of DCS system facilitate operations of phase/equipment module 515 and OPC-UA interface module 525. Phases in phase equipment module 515 are the building blocks for recipes, and when running batch recipes are sent as commands to the Equipment Module. The Equipment Module has pre-defined commands that control the instruments including start protocol, start recovery protocol and set parameters. OPC-UA Interface Module 525 maps the data between DCS system and each of the instruments blood processing device 122, magnetic bead processing device 120, electroporation system 118, freezer system 116, and incubator system 114. OPC network layer 536 connects various components of DCS system including, computing system 546, controllers 544, and instrument set including, blood processing device 122, magnetic bead processing device 120, electroporation system 118, freezer system 116, and incubator system 114 for seamless electronic communication.

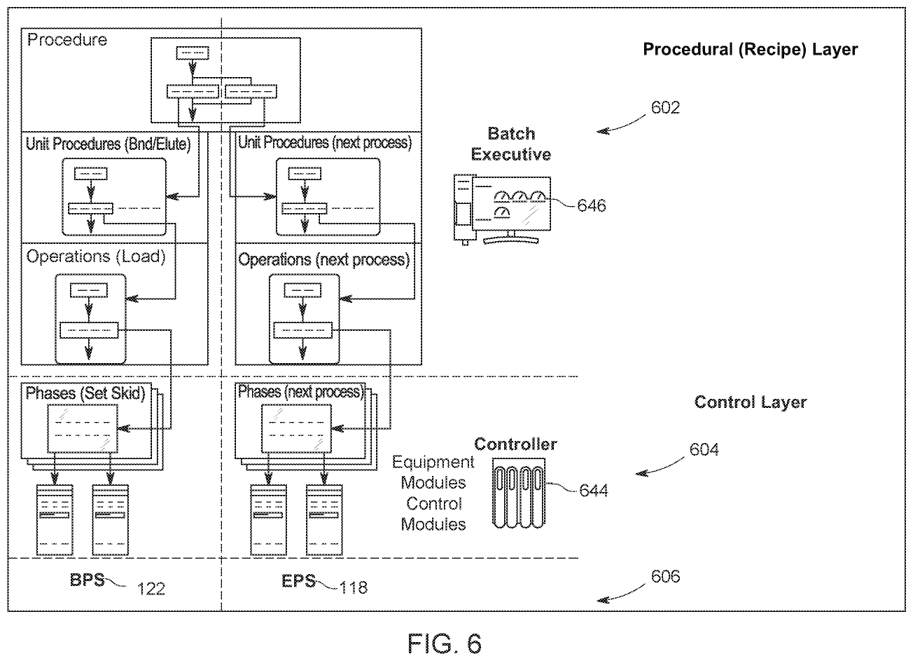

FIG. 6 depicts a flow chart for batch control data architecture for the bioprocess control system (BPCS) in embodiments of the disclosure. The architecture includes three main layers including, procedural (recipe)layer 602, control layer 604, and instrument layer 606. In the example shown in FIG. 6, blood processing system 122, and electroporation system 118 are shown, but the architecture can include other instruments including magnetic bead processing system 120, freezer system 116, and incubator system 114. Procedural layer 602 is controlled by computing system 646 and includes procedures that include unit operations. Further unit operations can include operations and operations can include phase instances. The control layer is 604 can be controlled by controller 644. Phase operations are loaded and executed as the operations proceeds.

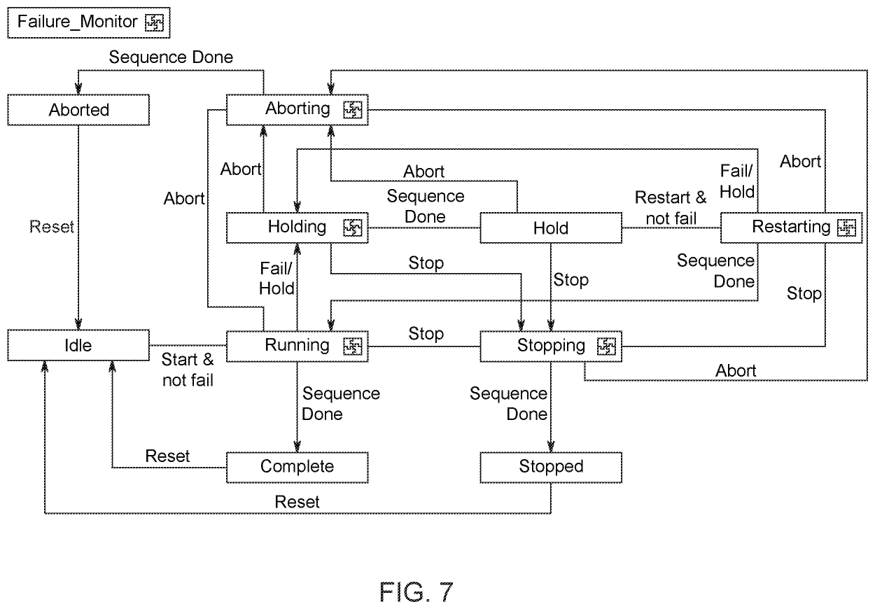

FIG. 7 depicts a schematic phase diagram for software modules for the bioprocess control system (BPCS) in embodiments of the disclosure. Phases are software modules that conform to batch standards (example ISA-88) and run on the controllers 644 as shown in FIG. 6. Phases have different states when running, including failure monitor, sequence done, aborted, reset, idle, holding, fail/hold, completer, stop, fail/hold etc. When phases are running on an instrument, the phase status, messages and prompts are displayed on the instrument display in the batch banner section (Example, 908F in FIG. 9A)

FIG. 8 illustrates a graphical user interface 800 displayed on a computing device 100 in accordance with illustrative embodiments. The graphical user interface 800, represents an overview screen and can be used to control the bioproduction system 10 by controlling the interconnected cell processing system 12 including bioprocessing instrument set 112. As described herein, in various embodiments, the bioprocessing instrument set 112 includes a blood processing system 122, a magnetic bead processing system 120, an electroporation system 118, optionally a freezing system 116, optionally an incubator system 114 and optionally a bioreactor/bioreactor pod. In embodiments, the interface 800 includes screen and display 802 (touch screen/display or other physical input) and graphical user interface 804 with primary selection area 806 and secondary selection area 808. Primary selection area 806 of graphical user interface 804 includes icons 805 for a blood processing system (BPS) 122 (“Rotea™”), a magnetic bead processing system (MBPS) 120 (“DynaCellect™”), an electroporation system (EPS) 118 (“Xenon”), a freezing system (FS) 116 (“Cryomed™”), and an incubator system (IS) 114 (“Incubator”). Access to operate and control operations of the blood processing system BPS 122 can be achieved by activating icon 805A. Similarly, access for operating magnetic bead processing system MBPS 120 can be achieved by activating icon 805B. Access to operate electroporation system EPS 118 can be achieved by activating icon 805C. Access for operating freezer system FS 114 can be obtained by activating icon 806D. Access for operating incubator system IS 116 can be obtained by activating icon 806. Secondary selection area 808 includes forward and backward arrows to navigate the user to previous or later user interfaces.

FIG. 9A illustrates a user interface 900 displayed on a client computing device 100 in accordance with example embodiments. In illustrative embodiments, user interface 900 can be obtained upon accessing icon 805A shown in FIG. 8. The user interface 900 is also an overview display for BPS and can be used to control blood processing system (BPS) 122. The user interface 900 can include a screen and display 902 (touch screen/display or other physical input) and a graphical user interface 904 with a primary selection area 906 and secondary selection areas 908A-E. The primary selection area 906 displays virtual input/output controls 910A-B and fluid flow paths 912 having fluid flow lines labeled as, A, C, D, E, F, G, and H in a kit to facilitate control functions of the blood processing system 122 by transmitting inputs or protocol recipes to a controller 108. The virtual input controls 910A can be actuated to skip a step, pause the run, or stop the run of a protocol or recipe that controls the flow of cells in a biological sample through fluid flow lines (A-H) of a fluid management kit and the rotational speed and flow of cells through a centrifuge of the blood processing system 122. The centrifuge can be a counterflow centrifuge. A virtual input/output control 910B can display or control the directional flow of cells and the rotational speed and orientation of a counterflow centrifuge in the blood processing system 122. The virtual fluid flow paths 912 having fluid flow lines labeled as, A, C, D, E, F, G, and H in the primary selection area 906 can display the valve position and an animation of the fluid flow routes that the cells will take as they pass through the fluid management kit of the blood processing system 122 to the centrifuge.

One or more secondary selection areas 908A-E of the graphical user interface 904 can include status, details of the workflows, and protocols for blood computing device 122. Secondary selection area 908A includes the instrument name (e.g., BPS 001 (i.e., Rotea™ 001)); the protocol state (e.g., running); Secondary selection area 908B includes the instrument status (e.g., run time, kit ID, door state, pump flow rate, mass centrifuged); Secondary selection area 908C includes the protocol status (e.g., protocol name, step number description, and time remaining); Secondary selection area 908D includes batch information (e.g., batch ID, Unit procedure, procedure). Further, secondary selection area 908D includes a ‘See batch list” icon, and actuating the “See batch list” icon outputs a display of batch list information. Additionally actuating the “See batch list” icon outputs, the batch faceplate to start/stop recipes. Secondary selection area 908E includes alerts/prompts and alerts/prompts that are displayed in the event of an instrument error or failure. In particular, the selection area 908E displays error messages only if an error exists and displays the active instrument error.

Secondary selection areas 908F, and 908G are located at the bottom of the user interface 904 and displays information regarding messages and prompts related to batch recipes, and equipment module, respectively. Further, the secondary selection area 908G includes an “instrument status” icon, and upon actuation the “instrument status” icon outputs a display of an equipment module faceplate.

The user interface 904 includes a menu button 914 located below the title bar and upon actuation provides links to other displays including the main display, and interface parameters display. Further, the user interface 904 includes an i-icon 916 located below the menu button 914, and actuating the i-icon 916 provides a tooltip to show the display name and revision.

Graphical user interface 904B as shown in FIG. 9B can be obtained by accessing menu button 914 shown in FIG. 9A. Graphical user interface 904B is also a main display for BPS and can be used to control blood processing system (BPS) 122. Graphical user interface 904B illustrates a direction of fluid flow 918 in fluid flow paths 912 for blood processing system (BPS) 122. As seen in FIG. 9B fluid flow paths have a plurality of interconnected fluid flow lines labeled as A, B, C, D, E, F, G, H. For example, the direction of fluid flow 918 can also highlight if the fluid flow is in forward or backward direction. Additionally, the direction of fluid flow 918 can also highlight if the fluid flow is through a combination of fluid line A and fluid line B or any other combination of fluid lines.

Graphical user interface 904C as shown in FIG. 9C can be obtained by accessing menu button 914 shown in FIG. 9A. Graphical user interface 904C is also an interface parameter display for BPS 122 and can be used to control and/or monitor blood processing system (BPS) 122. Graphical user interface 904C functions as a primary resource for diagnostic purposes or to see more detailed information of blood processing system 122. Graphical user interface 904C as shown in FIG. 9C provides information related to interface parameters for blood processing system (BPS) 122. Graphical user interface 904C includes a primary selection area 906C, including a plurality of selection areas, instrument status selection area 920C, protocol details selection area 922C, parameters selection area 924C, commands to instrument selection area 926C, and protocol list selection area 930. Instrument status selection area 920C provides information about blood processing instrument (BPS) 122, including name, model number, serial number, main firmware version, power firmware version, app version, uptime, DCS-PC-link, kit state, kit ID, door state, user level, pump rate, centrifuge speed, and pump direction. Protocol details selection area 922C provides information about blood processing system 122, including name, checksum, state, step name, time remaining, total steps, ended on, error raised, waiting ack, run log file and run log pdf. Parameters selection 924C provides information about parameters description, state, value related to blood processing system 122. Commands to instrument selection area provide information related to blood processing system including GUI user level, protocol ID, protocol state, recovery step No., Rescan kit, play/pause panel PB, and advance PB. Upon accessing the protocol list selection area 930 a pop-up window including a protocol list is opened as shown in FIG. 9C.

Graphical user interface 904D as shown in FIG. 9D can be obtained by accessing secondary selection area 908G as shown in FIG. 9A. Graphical user interface 904D is also an equipment module faceplate display for BPS 122 and can be used to control blood processing system (BPS) 122. Upon accessing secondary selection area 908G a BPS equipment module pop-up window 932C is displayed as shown is as shown in FIG. 9D. Equipment module pop-up window 932C includes options for selecting a command drop-down box, run button, release button, information about the current command being run, current state of the command, current owner of the module, alarm window and a set of selection icons 934C at the bottom portion. Upon accessing the settings icon in the set of selection icons 934C, an equipment module pop-up window 936C for BPS equipment module is displayed. Equipment module pop-up window 936C includes four tabs which can be used to select the different types of parameters, Operator, Tuning, Display, and Batch Recipe. BPS Equipment module pop-up window 932C allows user to stop/Run Protocol/Run Recovery from Step/Run Recovery Single Step/Monitor Protocol/Recovery Control protocol run on blood processing system 122. The parameters shown on the Operator parameter tab can be modified by a user with operator privileges, and they are used to control how the protocol runs the operation of blood processing system BPS 122, in a bioprocessing instrument set 112. The parameters shown on the Tuning parameter tab can be modified only by a user with configuration privileges, and they are used to control how the user interface functions. The parameters shown on the Display parameter tab are internally calculated for display only and cannot be modified. The parameters shown on the batch recipe parameter tab are updated by a phase running as part of a batch recipe and cannot be modified, and they can be used to control how the protocol runs.

FIG. 10A illustrates a user interface 1000A displayed on a client computing device 100 in accordance with example embodiments. The user interface 1000A can be obtained by accessing icon 805A in FIG. 8, and can be used to control and/or monitor a magnetic bead processing system 120. The user interface 1000A is also referred as an overview display for magnetic bead processing system 120. The user interface 1000A can include a screen and display 1002A (touch screen/display or other physical input) and a graphical user interface 1004A with a primary selection area 1006A and secondary selection areas 1008A-E. The primary selection area 1006A displays fluid flow paths 1010 (A-K) in a kit to facilitate control and monitor functions of the magnetic bead processing system 120 by transmitting inputs or protocol recipes to a controller 108. The virtual fluid flow paths 1010 (A-K) in the primary selection area 1006 can display the valve position and an animation of the fluid flow routes that the cells will take as they pass through the bead processing system 120.

One or more secondary selection areas 1008A-F of the graphical user interface 1004 can include status, details of the workflows, and protocols for electroporation system 118. Secondary selection area 1008A includes the instrument name (e.g., CTI-MBPS); the protocol state (e.g., running, stopped/Idle); Secondary selection area 1008B includes the instrument and protocol status (e.g., Instrument status, Run ID, Kit ID, Rocker state, Magnet state, Protocol status, Protocol name, Protocol type, Protocol status, Step number, step time remaining, Description etc.); Secondary selection area 1008C includes batch information including, batch ID, Operation, Unit procedure, and procedure details of each run processed in the bead processing system 120 and batch list details; Secondary selection area 1008D includes alerts for device errors. Secondary selection areas 1008E, and 1008F are located at the bottom of the user interface 1004 and display information regarding messages and prompts related to batch recipes, and the status of the equipment module, respectively.

The user interface 1004A further includes a menu button 1016A located below the title bar and upon actuation provides links to other displays including the diagnostic display and overview display. Further, user interface 1004A includes an icon 1018A located below the menu button 1016A, and actuating the i-icon 1018A provides a tooltip to show the display name and revision.

Graphical user interface 1004B as shown in FIG. 10B can be obtained by accessing secondary selection area 1008F as shown in FIG. 10A. Graphical user interface 1004B is also an equipment module faceplate display for MBPS 120 and can be used to control and/or monitor magnetic bead processing system (MBPS) 120. Upon accessing secondary selection area 1008F a MBPS equipment module pop-up window 1022 is displayed as shown in FIG. 10B. Equipment module pop-up window 1022 includes options for select a command drop down box, run button, release button, information about current command being run, current state of the command, current owner of the module, alarm window and a set of selection icons 1024 at the bottom portion. Upon accessing the settings icon in the set of selection icons 1024, an equipment module pop-up window 1026 for BPS equipment module is displayed. Equipment module pop-up window 1022 allows user to Stop protocol/Run Protocol/Run Recovery/Monitor Protocol/Recovery Control protocol run on magnetic bead processing system 120. Equipment module pop-up window 1026 includes four tabs which can be used to select the different types of parameters, Operator, Tuning, Display, and Batch Recipe. The parameters shown on the Operator parameter tab can be modified by a user with operator privileges, and they are used to control how the protocol runs the operation of the magnetic bead processing system MBPS 120, in a bioprocessing instrument set 112. The parameters shown on the Tuning parameter tab can be modified only by a user with configuration privileges, and they are used to control how the user interface functions. The parameters shown on the Display parameter tab are internally calculated for display only and cannot be modified. The parameters shown on the batch recipe parameter tab are updated by a phase running as part of a batch recipe and cannot be modified, and they can be used to control how the protocol runs.

FIGS. 11A and 11B illustrate user interfaces 1100A, 1100B, respectively, displayed on a client computing device 100 in embodiments of the disclosure. The user interfaces 1100A, 1100B can be obtained by accessing icon 805C in FIG. 8, and can be used to control and/or monitor the electroporation system 118. FIG. 11A illustrates screenshot 1100A of the user interface when the electroporation system is in operational or running mode. Similarly, FIG. 11B illustrates screenshot 1100B of the user interface when the electroporation system is set ready for a run.

The user interface 1100A can include a screen and display 1102A (touch screen/display or other physical input) and a graphical user interface 1104A with a primary selection area 1106A and secondary selection areas 1108A-G. The primary selection area 1106A displays one or more virtual input/output controls (1110A, 1112A, 1114A, 1116A, 1118A) to facilitate control functions of the electroporation system 118 by transmitting inputs or protocol recipes to a controller 108. A virtual output control 1110A displays an amount of volume remaining, a virtual output control 1112A displays elapsed time, and a virtual output control 1114A displays the amount of volume completed. A virtual output control 1116A displays a time remaining for a particular step in a dial format. Further, a virtual output control 1118A displays paused time during the run or cycle.

One or more secondary selection areas 1108A-F of the graphical user interface 1104A can include status, details of the workflows, and protocols for electroporation system 118. Secondary selection area 1108A includes the instrument name (e.g., CTI_EPS); the protocol state (e.g., running, stopped/Idle); Secondary selection area 1108B includes the instrument and protocol status (e.g., Instrument status, door state, OPC UA status, Protocol Name, SS run state, MS run state, MS run details, Volume, Temperature, Pulse voltage, Pulse width, Pulse delay, Buffer type etc.); Secondary selection area 1108C includes batch information including, batch ID, Operation, Unit procedure, and procedure details of each run processed in the electroporation system 118 and batch list details; Secondary selection area 1108D includes alerts for device errors. Secondary selection areas 1108E, and 1108F are located at the bottom of user interface 1104A and display information regarding messages and prompts related to batch recipes, and the status of the equipment module, respectively.

The user interface 1104A includes a menu button 1120A located below the title bar and upon actuation provides links to other displays including the diagnostic display and overview display. Further, user interface 1104A includes an icon 1122A located below the menu button 1120A and actuating the i-icon 1118A provides a tooltip to show the display name and revision.

Graphical user interface 1104B as shown in FIG. 11B can be obtained by accessing secondary selection area 1108F as shown in FIG. 11A. Graphical user interface 1104B is also referred to as an equipment module faceplate display for EPS 118 and can be used to control electroporation system (EPS) 118. Upon accessing secondary selection area 1108F a EPS equipment module pop-up window 1124 is displayed as shown in FIG. 11B. Equipment module pop-up window 1124 includes options for select a command drop down box, run button, release button, information about current command being run, current state of the command, current owner of the module, alarm window and a set of selection icons 1126 at the bottom portion. Upon accessing the settings icon in the set of selection icons 1126, an equipment module pop-up window 1128 for EPS equipment module is displayed. Equipment module pop-up window 1124 allows user to Stop Protocol/Single Shot Protocol/Multi Shot Protocol/Monitor Single Shot/Monitor Extraction/Monitor Multi Shot protocol run on electroporation system 118. Equipment module pop-up window 1128 includes four tabs which can be used to select the different types of parameters, Operator, Tuning, Display, and Batch Recipe. The parameters shown on the Operator parameter tab can be modified by a user with operator privileges, and they are used to control how the protocol runs the operation of electroporation system EPS 116, in a bioprocessing instrument set 112. The parameters shown on the Tuning parameter tab can be modified only by a user with configuration privileges, and they are used to control how the user interface functions. The parameters shown on the Display parameter tab are internally calculated for display only and cannot be modified. The parameters shown on the batch recipe parameter tab are updated by a phase running as part of a batch recipe and cannot be modified, and they can be used to control how the protocol runs.

FIGS. 12A, FIG. 12B, and FIG. 12C illustrate user interfaces 1200A, 1200B, 1200C respectively, and are displayed on a client computing device 100 in accordance with example embodiments. The user interfaces 1200A, 1200B, 1200C can be used to control and/or monitor the incubator system 114. While FIG. 12A illustrates user interface 1200A when the incubator system is in operational or running mode, FIG. 12B and FIG. 12C illustrate user interfaces 1200B and 1200C when the incubator system is in idle mode. While user interface 1200A can be referred to as an overview display for Incubator system 114, user interface 1200B can be referred to as a main display, and user interface 1200C can be referred to as an equipment module faceplate display for incubator system 114.

User interface 1200A can be obtained by accessing icon 805D in FIG. 8, and can include a screen and display 1202A (touch screen/display or other physical input) and a graphical user interface 1204A with a primary selection area 1206A and secondary selection areas 1208A-G. The primary selection area 1206A displays one or more virtual input/output controls (1210A, 1212A, 1214A, 1216A) to facilitate control and/or monitor functions of the incubator system 114 by transmitting inputs or protocol recipes to a controller 108. A virtual input/output control 1210A displays a temperature setpoint, a virtual output control 1212A displays a PV temperature, and a virtual output control 1214A displays time remaining. Monitoring of a sample temperature versus time in an operational run is very critical for the integrity of biological samples processed in the incubator 114. A graphical output 1216A displays the variation of incubator chamber temperature with time in the incubator system.

One or more secondary selection areas 1208A-G of the graphical user interface 1204 can include status, details of the workflows, and protocols for incubator system 116. Secondary selection area 1208A includes the instrument name (e.g., IS2745); the protocol state (e.g., running, stopped/Idle); Secondary selection area 1208B includes the instrument parameter status (e.g., Actual temperature, Actual CO2 concentration, Actual water level, Low Humidity, Door status, HEPA Status, etc.); Secondary selection area 1208C includes the status of Steri-run program and Auto-run program. Secondary selection area 1208D includes batch information including, batch ID, Operation, Unit procedure, and procedure details of each run processed in the incubator system 114; Secondary selection area 1208E includes alerts for device errors. Secondary selection areas 1208F, and 1208G are located at the bottom of the user interface 1204A and display information regarding messages and prompts related to batch recipes, and status of the equipment module, respectively.

The user interface 1204A includes a menu button 1218A located below the title bar and upon actuation provides links to other displays including the main display and diagnostic display. Further, the user interface 1204A includes an icon 1220A located below the menu button 1218A, and actuating the i-icon 1220A provides a tooltip to show the display name and revision.

Graphical user interface 1204B shown in FIG. 12B is similar to user interface 1204A and includes one or more virtual input/output controls 1222, 1224, 1226 to facilitate monitor and/or control functions of the incubator system 114 by transmitting inputs or protocol recipes to a controller 108. A virtual output control 1222 displays a live sample temperature and in the incubator chamber. A virtual output control 1224 displays a live CO2 concentration in the incubator chamber. A virtual output control 1226 displays a live relative humidity in the incubator chamber. Optionally a virtual output control to monitor oxygen (02) content can also be included in graphical user interface 1204A.

Graphical user interface 1204C as shown in FIG. 12C can be obtained by accessing secondary selection area 1208G as shown in FIG. 12A. Graphical user interface 1204C is also referred to as an equipment module faceplate display for IS 116 and can be used to control incubator system (IS) 114. Upon accessing secondary selection area 1208G an EPS equipment module pop-up window 1228 is displayed as shown in FIG. 12C. Optionally, equipment module pop-up window 1228 can include options for selecting a command drop down box, run button, release button, information about current command being run, current state of the command, current owner of the module, alarm window and a set of selection icons 1230 at the bottom portion. Upon accessing the settings icon in the set of selection icons 1230, an equipment module pop-up window 1232 for IS equipment module is displayed. Optionally, equipment module pop-up window 1232 can contain any alarms configured and any parameters to be displayed on the operator interface. Equipment module pop-up window 1232 includes four tabs which can be used to select the different types of parameters, Operator, Tuning, Display, and Batch Recipe. Optionally, the parameters shown on the Operator parameter tab can be modified by a user with operator privileges, and they are used to control how the protocol runs the operation of incubator system IS 114, in a bioprocessing instrument set 112. Optionally, the parameters shown on the Tuning parameter tab can be modified only by a user with configuration privileges, and they are used to control how the user interface functions. Optionally, the parameters shown on the Display parameter tab are internally calculated for display only and cannot be modified. Optionally, the parameters shown on the batch recipe tab can be modified by a user with operator privileges, and they can be used to control how the protocol runs.

FIG. 13A and FIG. 13B illustrate a user interface 1300 displayed on a client computing device 100 in accordance with example embodiments. The user interface 1300 can be obtained by accessing icon 805E in FIG. 8, and can be used to control the freezing system 116 for freezing and preserving the output or end-product of the electroporation system 118. The user interface 1300 can include a screen and display 1302 (touch screen/display or other physical input) and a graphical user interface 1304 with a primary selection area 1306 and secondary selection areas 1308A-E. The primary selection area 1306 displays one or more virtual input/output controls (1310, 1312, 1314) to facilitate control functions of the freezing system 116 by transmitting inputs or protocol recipes to a controller 108. A virtual output control 1310 displays a live sample temperature and live chamber temperature. Monitoring of a sample temperature versus chamber temperature is very critical for the integrity of biological samples stored in the freezing unit 116. A graphical output 1312 displays a trend data for sample and chamber temperatures, Further control icons 1314 can be actuated to advance a step, stop a cooling recipe that controls the cooling of samples in a cryo-chamber of freezing system 116, or provide access for manual control. Furthermore, manual control includes cool active, cool plus active, warm active, and warming modes to regulate the temperature of the sample and chamber.

One or more secondary selection areas 1308A-E of the graphical user interface 1304 can include status, details of the workflows, and protocols for freezing system 116. Secondary selection area 1308A includes the instrument name (e.g., FSXXXX); the protocol state (e.g., running, stopped/Idle); Secondary selection area 1308B includes the instrument status (e.g., Status, Profile name, manual command, Step number, OPC UA Link status, and description); Secondary selection area 1308C includes batch information (e.g., batch ID, Operation, Unit procedure, procedure). Further, secondary selection area 1308C includes a ‘See batch list” icon and upon actuation the “See batch list” icon outputs a display of batch list information. Additionally actuating the “See batch list” icon outputs, the batch faceplate to start/stop recipes. Secondary selection area 1308D includes alerts/prompts and alerts/prompts that are displayed in the event of an instrument error or failure. In particular, the selection area 308D displays error messages only if an error exists and displays the active instrument error.

Secondary selection areas 1308E, and 1308F are located at the bottom of the user interface 1304 and displays information regarding messages and prompts related to batch recipes, and equipment module, respectively. Further, the secondary selection area 1308F includes an “instrument status” icon, and upon actuation the “instrument status” icon outputs a display of an equipment module faceplate.

The user interface 1304 includes a menu button 1316 located below the title bar and upon actuation provides links to other displays including the diagnostic display and overview display. Further, the user interface 1304 includes an icon 1318 located below the menu button 1316 and actuating the i-icon 1318 provides a tooltip to show the display name and revision.

Graphical user interface 1304B as shown in FIG. 13B can be obtained by accessing menu button 1316 shown in FIG. 13A. Graphical user interface 1300B is also a main display for FS and can be used to control freezer system (FS) 116. Graphical user interface 1304B is very similar to overview display 1304 shown in FIG. 13A.

Graphical user interface 1304C as shown in FIG. 13C can be obtained by accessing secondary selection area 1308F as shown in FIG. 13A. Graphical user interface 1304C is also an equipment module faceplate display for FS 116 and can be used to control the freezer system (FS) 116. Upon accessing secondary selection area 1308F a FS equipment module pop-up window 1322 is displayed as shown is as shown in FIG. 13C. Equipment module pop-up window 1322 includes options for selecting a command drop-down box, run button, release button, information about the current command being run, the current state of the command, current owner of the module, alarm window and a set of selection icons 1324 at the bottom portion. Upon accessing the settings icon in the set of selection icons 1324, an equipment module pop-up window 1326 for FS equipment module is displayed. Equipment module pop-up window 1322 allows user to Stop Profile/Run Profile/Monitor Profile protocol run on freezer system 116. Equipment module pop-up window 1326 includes four tabs that can be used to select the different types of parameters, Operator, Tuning, Display, and Batch Recipe. The parameters shown on the Operator parameter tab can be modified by a user with operator privileges, and they are used to control how the protocol runs the operation of freezer system FS 116, in a bioprocessing instrument set 112. The parameters shown on the Tuning parameter tab can be modified only by a user with configuration privileges, and they are used to control how the user interface functions. The parameters shown on the Display parameter tab are internally calculated for display only and cannot be modified. The parameters shown on the batch recipe parameter tab are updated by a phase running as part of a batch recipe and cannot be modified, and they can be used to control how the protocol runs.

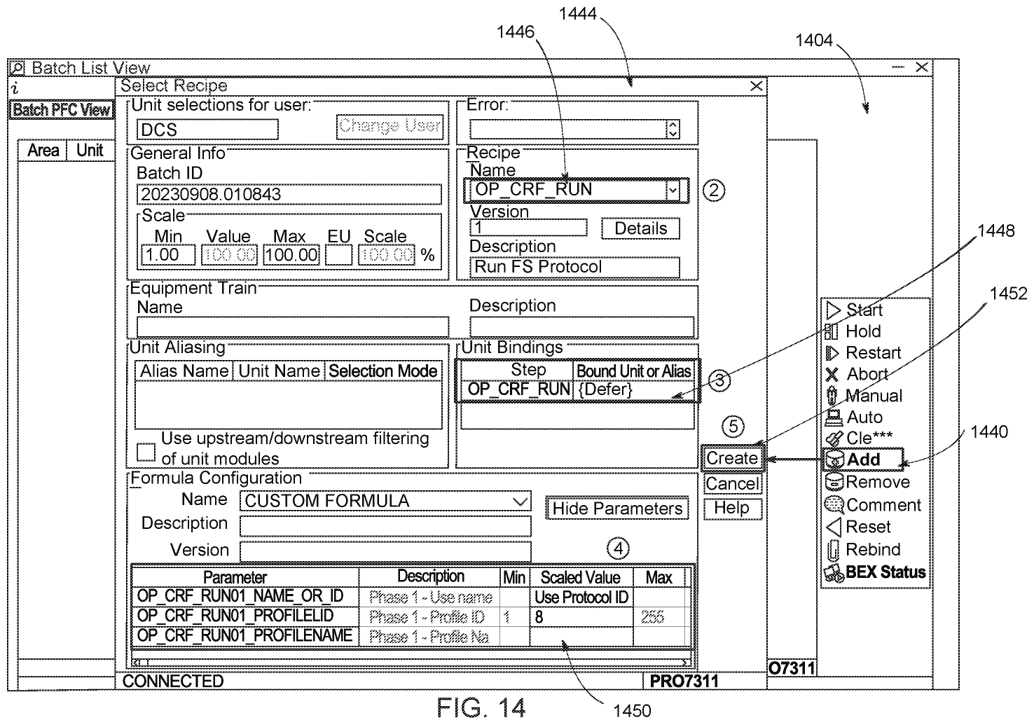

FIG. 14 illustrates an example graphical user interface 1404 for running batch recipes on one or more specific bioprocessing instrument(s) 114, 116, 118, 120, 122 of the bioprocessing instrument set 112 and configured to receive a virtual input from a user, as well as readable instrument and process parameter outputs (e.g., a device or process state) for controlling operation and/or monitoring of a bioproduction process (e.g., cell therapy manufacturing workflow) performed by a bioprocessing instrument(s) 114, 116, 118, 120, 122, composing a bioprocessing instrument set 112. In the example shown in FIG. 14, user interface 1404 can be obtained by accessing the secondary selection area 908F for blood processing system BPS, as shown in FIG. 9A. Similarly respective user interfaces for running batch recipes can be obtained by accessing respective secondary selection areas in respective user interfaces for each of magnetic bead processing system 120, electroporation system 118, freezer system 116, and incubator system 114.

Upon accessing ‘Add button’ 1440 on user interface 1404 customization of a batch recipe can be started by obtaining pop-up window 1444. Further by accessing drop-down menus 1446, 1448, and modifying parameters in formula configuration 1450 a batch recipe for an instrument of interest can be defined by the user. Finally, by accessing ‘Create’ button 1452 users can complete creating a batch recipe. Optionally, a list of batch recipes created by the user for the instrument of interest can be viewed in the batch list view mode of user interface 1404.

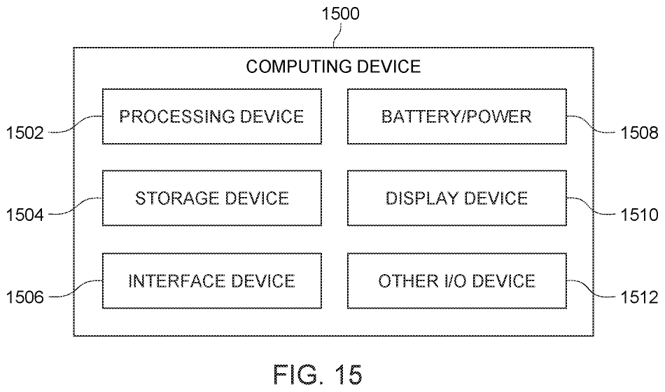

FIG. 15 depicts a block diagram of an example computing device 1500 that can perform some or all operations of a bioproduction system, client computing device(s), switch(es) and/or controller(s) in accordance with the example embodiments. The example bioprocess control systems, modules, and libraries, including bioprocessing instrument(s) 114, 116, 118, 120, 122, client computing devices 100 with user interfaces 100, computing devices 104 and controllers 108, disclosed herein can include or be implemented by one or more computing devices. In some embodiments, the example client computing devices 100, computing devices 104, and controllers 108 include a single computing device 1500 or multiple computing devices 1500. Further, as discussed below, a computing device 1500 (or multiple computing devices 1500) that implements the example bioprocess control systems, modules and libraries can be part of one or more bioprocessing instrument(s) 114, 116, 118, 120, 122, client computing devices 100 with user interfaces 100, computing devices 104 and controllers 108, a user's local computing device, a service provider's local computing device, or a remote computing device. The client computing devices 100, computing devices 104, and controllers 108 can also be contained in a unitary computing system or server with a user interface or distributed servers and systems.

The computing device 1500 of FIG. 15 is illustrated as having a number of components, but any one or more of these components may be omitted or duplicated, as suitable for the application and setting. In some embodiments, some or all of the components included in the computing device 1500 can be attached to one or more motherboards and enclosed in a housing (e.g., including plastic, metal, and/or other materials). In some embodiments, some these components may be fabricated onto a single system-on-a-chip (SoC) (e.g., an SoC may include one or more processing devices 1504). Additionally, in various embodiments, the computing device 1500 may not include one or more of the components illustrated in FIG. 15, but may include interface circuitry (not shown) for coupling to the one or more components using any suitable interface (e.g., a Universal Serial Bus (USB) interface, a High-Definition Multimedia Interface (HDMI) interface, a Controller Area Network (CAN) interface, a Serial Peripheral Interface (SPI) interface, an Ethernet interface, a wireless interface, or any other appropriate interface). For example, the computing device 1500 may not include a display device 1510, but may include display device interface circuitry (e.g., a connector and driver circuitry) to which a display device 1510 may be coupled.

The computing device 1500 can include a processing medium or device 1502 (e.g., one or more processing devices). As used herein, the term “processing device” refer to any device or portion of a device that processes electronic data from registers and/or memory to transform that electronic data into other electronic data that may be stored in registers and/or memory. The processing device 1502 can include one or more digital signal processors (DSPs), application-specific integrated circuits (ASICs), central computing devices (CPUs), graphics computing devices (GPUs), crypto processors (specialized processors that execute cryptographic algorithms within hardware), server processors, or any other suitable processing devices.

The computing device 1500 can also include a storage device 1504 (e.g., one or more storage devices). The storage device 1504 can include one or more memory devices such as random-access memory (RAM) (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some embodiments, the storage device 1504 can include memory that shares a die with a processing device 1502. In such an embodiment, the memory can be used as cache memory and can include embedded dynamic random-access memory (eDRAM) or spin transfer torque magnetic random-access memory (STT-MRAM), for example. In some embodiments, the storage device 1504 can include non-transitory computer-readable media having instructions thereon that, when executed by one or more processing devices (e.g., the processing device 1502), cause the computing device 1500 to perform any appropriate ones of or portions of the methods and operations disclosed herein.

The computing device 1500 can include an interface device 1506 (e.g., one or more interface devices 1506). The interface device 1506 can include one or more communication chips, connectors, and/or other hardware and software to govern communications between the computing device 1500 and other computing devices. For example, the interface device 1506 can include circuitry for managing wireless communications for the transfer of data to and from the computing device 1500. The term “wireless” and its derivatives are used to describe circuits, devices, systems, methods, techniques, communications channels, etc., that can communicate data using modulated electromagnetic radiation through a nonsolid medium. The term does not imply that the associated devices do not contain any wires, although in some embodiments they might not. Circuitry included in the interface device 1506 for managing wireless communications can implement any of a number of wireless standards or protocols, including but not limited to Institute for Electrical and Electronic Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some embodiments, circuitry included in the interface device 4006 for managing wireless communications may operate in accordance with a Global System for Mobile Communication (GSM), General Packet Radio Service (GPRS), Universal Mobile Telecommunications System (UMTS), High-Speed Packet Access (HSPA), Evolved HSPA (E-HSPA), or LTE network. In some embodiments, circuitry included in the interface device 4006 for managing wireless communications can operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). In some embodiments, circuitry included in the interface device 4006 for managing wireless communications can operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some embodiments, the interface device 4006 can include one or more antennas (e.g., one or more antenna arrays) to receipt and/or transmission of wireless communications.

In some embodiments, the interface device 1506 (e.g., computing device) includes one or more processors and/or memories, as well as circuitry for managing input/output communication via wired/wireless communications, such as electrical, optical, Wi-Fi, or any other suitable communication protocols. For example, the interface device 1506 can include circuitry to support communications in accordance with Ethernet technologies. In some embodiments, the interface device 1506 can support both wireless and wired communication and/or may support multiple wired communication protocols and/or multiple wireless communication protocols. For example, a first set of circuitries of the interface device 1506 can be dedicated to shorter-range wireless communications such as Wi-Fi or Bluetooth, and a second set of circuitry of the interface device 1506 can be dedicated to longer-range wireless communications such as global positioning system (GPS), EDGE, GPRS, CDMA, WiMAX, LTE, EV-DO, or others. In some embodiments, a first set of circuitries of the interface device 1506 can be dedicated to wireless communications, and a second set of circuitries of the interface device 1506 can be dedicated to wired communications.

The computing device 1500 can include battery/power circuitry 1508. The battery/power circuitry 1508 can include one or more energy storage devices (e.g., batteries or capacitors) and/or circuitry for coupling components of the computing device 8000 to an energy source separate from the computing device 1500 (e.g., AC line power).

The computing device 1500 can include a display device 1510 (e.g., multiple display devices). The display device 1510 can include any visual indicators, such as a heads-up display, a computer monitor, a projector, a touchscreen display, a liquid crystal display (LCD), a light-emitting diode display, or a flat panel display.

The computing device 1500 can include other input/output (I/O) devices 1512. The other I/O devices 1512 can include one or more audio output devices (e.g., speakers, headsets, earbuds, alarms, etc.), one or more audio input devices (e.g., microphones or microphone arrays), location devices (e.g., GPS devices in communication with a satellite-based system to receive a location of the computing device 1500, as known in the art), audio codecs, video codecs, printers, sensors (e.g., thermocouples or other temperature sensors, humidity sensors, pressure sensors, vibration sensors, accelerometers, gyroscopes, etc.), image capture devices such as cameras, keyboards, cursor control devices such as a mouse, a stylus, a trackball, or a touchpad, bar code readers, Quick Response (QR) code readers, or radio frequency identification (RFID) readers, for example.

The computing device 1500 can have any suitable form factor for its application and setting, such as a handheld or mobile computing device (e.g., a cell phone, a smartphone, a mobile internet device, a tablet computer, a laptop computer, a netbook computer, an Ultrabook computer, a personal digital assistant (PDA), an ultra-mobile personal computer, etc.), a desktop computing device, or a server computing device or other networked computing components.

In various embodiments, the system includes one or more machine learning modules. Such modules may be utilized to optimize parameters of one or more of the bioprocessing instruments for example to generate and/or optimize protocols, data storage, event/alarm chronicling, batch control, functional operation between instruments and the like.

In certain aspects the present disclosure provides a system for bioprocessing that includes a computing device comprising one or more processors, and a memory, wherein the memory stores instructions that, when executed by the one or more processors, cause the computing device to perform any of the methods as disclosed herein. For example, the memory can store one or more weights associated with one or more trained machine learning models including, for example, one or more trained neural networks, as disclosed herein. The term “weights” as used herein in reference to a neural network, refers to all parameter values and network structure definitions necessary to propagate input data through the neural network in order to obtain an output value.

The system embodiments disclosed herein may achieve improved performance relative to conventional approaches. As such, in embodiments, this invention utilizes a machine learning model, for example a neural network that, combined with an integrated, fast computational architecture, allows for operational improvement of bioprocessing instruments, individually or as a bioprocessing instrument set and/or system.

Various methods and systems of the embodiments disclosed herein may improve upon conventional approaches to achieve the technical advantages of higher throughput, more exact algorithms, and faster, more robust processing by making use of a machine learning model, for example a trained neural network that allows for real-time processing of data, and displaying of representations of bioprocessing data. Such technical advantages are not achievable by routine and conventional approaches, and all users of systems including such embodiments may benefit from these advantages, for example, by assisting the user in the performance of a technical task, such as real-time high-throughput bioprocessing, by means of a guided human-machine interaction process. The technical features of the embodiments disclosed herein are thus decidedly unconventional in the field of cell sorter, as are the combinations of the features of the embodiments disclosed herein. The present disclosure thus introduces functionality that neither a conventional computing device, nor a human, could perform. As used herein, the term ‘weights,’ in reference to a neural network, refers to all parameter values and network structure definitions necessary to propagate input data through the neural network to obtain an output value.

Various alterations and/or modifications of the inventive features illustrated herein and additional applications of the principles illustrated herein, which would occur to one skilled in the relevant art and having possession of this disclosure, can be made to the illustrated embodiments without departing from the spirit and scope of the invention as defined by the claims and are to be considered within the scope of this disclosure. Thus, while various aspects and embodiments have been disclosed herein, other aspects and embodiments are contemplated. While several methods and components similar or equivalent to those described herein can be used to practice embodiments of the present disclosure, only certain components and methods are described herein.

It will also be appreciated that systems, processes, and/or products according to certain embodiments of the present disclosure may include, incorporate, or otherwise comprise properties features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features without necessarily departing from the scope of the present disclosure.

The present disclosure may be embodied in other specific forms without departing from its spirit or essential characteristics. The embodiments described are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

1. A bioprocess control system to be used in connection with a bioprocessing instrument set comprising:
a computing device having a display and a processor disposed in connection with a memory, the display having a graphical user interface for each bioprocessing instrument of the bioprocessing instrument set configured in input/output communication with each bioprocessing instrument, each graphical user interface comprising a primary selection area configured to receive a virtual input from a user, and a secondary selection area having a virtual output,
wherein the memory comprises computer-readable instructions executable by the processor, which when executed cause the computing device to communicate with a bioprocessing instrument of the bioprocessing instrument set to control operation of the bioprocessing instrument dependent on the virtual input, and display a virtual output in the primary selection area or the secondary selection area, the virtual output being dependent on data received from the bioprocessing instrument.

2. The bioprocess control system of claim 1, wherein the virtual output indicates a device state or process state.

3. The bioprocess control system of claim 1, wherein the computing device is configured in input/output communication with at least one secondary computing device, the at least one secondary computing device having a memory storing instructions for controlling operation of one or more bioprocessing instruments of the bioprocessing instrument set.

4. The bioprocess control system of claim 3, wherein the at least one secondary computing device is communicatively configured in communication with the computing device and the one or more bioprocessing instruments.

5. The bioprocess control system of claim 4, wherein the computing device is communicatively configured in communication with the one or more bioprocessing instruments through the at least one secondary computing device and through at least one intermediary controller and/or network switch.

6. The bioprocess control system of claim 1, wherein the computing device is communicatively configured in communication with one or more bioprocessing instruments through at least one intermediary controller and/or network switch.

7. The bioprocess control system of claim 1, wherein the bioprocessing instrument set comprises at least 2 or more bioprocessing instruments selected from:
a blood processing system,
a magnetic bead processing system,
an electroporation system,
a freezing system,
an incubator system, and
a fill and finish system.

8. (canceled)

9. The bioprocess control system of claim 1, further comprising a machine learning module operable to perform analysis of data using a machine learning process.

10. The bioprocess control system of claim 9, wherein the machine learning process comprises a neural network.

11. The bioprocess control system of claim 10, wherein the neural network is a feedforward network or a deep neural network.

12. A system comprising:
a bioprocessing instrument set having at least 3 bioprocessing instruments selected from:
a blood processing system,
a magnetic bead processing system,
an electroporation system,
a freezing system,
an incubator system, and
a fill and finish system; and
a computing device comprising:
a processor; and
a memory storing computer-readable instructions executable by the processor, causing the computing device to:
display, on a display of the computing device, a graphical user interface comprising a primary selection area having at least one virtual input controlling operation of the bioprocessing instruments.

13. (canceled)

14. (canceled)

15. The system of claim 12, wherein the bioprocessing instrument set comprises:
a blood processing system,
a magnetic bead processing system,
an electroporation system,
a freezing system,
an incubator system, and
a fill and finish system.

16. (canceled)

17. (canceled)

18. The system of claim 15, wherein the graphical use interface further comprises a secondary selection area having at least one virtual output indicating a process state or a device state of one or more of the bioprocessing instruments.

19. The system of claim 18, wherein the secondary selection area has at least one virtual output indicating the process state or the device state of each of the bioprocessing instruments.

20. The system of claim 18, wherein the virtual input initiates a protocol controlling operation of one or more of the bioprocessing instruments, skips a step in the protocol controlling one or more of the bioprocessing instruments, temporarily pauses the protocol controlling the operation of one or more of the bioprocessing instruments, or permanently stops the protocol controlling the operation of one or more of the bioprocessing instruments.

21. The system of claim 12, wherein the memory of the computing device stores computer-readable instructions executable by the processor, to control operation of each bioprocessing instrument of the bioprocessing instrument set.

22. The system of claim 21, wherein the memory of the computing device stores computer-readable instructions executable by the processor, to cause the bioprocessing instruments to perform a series of operations to generate a population of genetically modified cells from a cell sample comprising a homogeneous population of non-genetically modified cells, or a heterogeneous population of non-genetically modified cells.

23-26. (canceled)

27. The system of claim 21, wherein each bioprocessing instrument comprises:
a memory storing computer-readable instructions executable by actuation of the at least one virtual input of the graphic user interface, the instructions operable to control operation of the individual bioprocessing instrument.

28-127. (canceled)

128. A method of generating genetically modified cells comprising:
providing a cell sample comprising live cells;
preparing the cell sample by centrifugation;
generating an enriched population of live cells by magnetic separation;
genetically modifying the enriched population of live cells via a transfection process comprising electroporation to generate a population of genetically modified cells;
incubating the population of genetically modified cells; and
washing the incubated cells,
wherein the method is performed using the bioprocess control system of claim 1, and wherein the method is performed under functionally-closed and sterile conditions.

129-134. (canceled)
